(19) United States
(12) Patent Application Publication
(10) Pub. No.: US 2025/0246009 A1
(43) Pub. Date: Jul. 31, 2025

(54) MULTI-MODAL PAIR MATCHING FOR A MULTI-MODAL MACHINE LEARNING MODEL LEARNING PROCESS

(71) Applicant: Recursion Pharmaceuticals, Inc., Salt Lake City, UT (US)

(72) Inventors: Jason Siyanda Hartford, Montreal (CA); Quanhan Xi, Port Moody (CA)

(21) Appl. No.: 18/672,492

(22) Filed: May 23, 2024

Related U.S. Application Data
(60) Provisional application No. 63/626,807, filed on Jan. 30, 2024.

(51) Int. Cl. G06V 20/69; G06V 10/74; G06V 10/774; G06V 10/776; G16B 25/10

(52) U.S. Cl. CPC G06V 20/698; G06V 10/761; G06V 10/774; G06V 10/776; G16B 25/10

(57) ABSTRACT

The present disclosure relates to systems, non-transitory computer-readable media, and methods that generate machine-learning training pairs across disparate modalities for a multi-modal machine learning model learning process. Indeed, in one or more implementations, the disclosed systems generate a first set of perturbation classification scores from a first data modality using a first classification model and a second set of perturbation classification scores from a second data modality using a second classification model. For instance, the disclosed systems compare the first set of perturbation classification scores with the second set of perturbation classification scores to determine a plurality of similarity measures. Moreover, in some instances, the disclosed systems identify pairs of data samples across the first data modality and the second data modality for a multi-modal machine learning model learning process using the pairs of data samples.

### CROSS REFERENCE TO RELATED APPLICATIONS

This application claims the benefit of and priority to U.S. Provisional Application No. 63/626,807, filed Jan. 30, 2024. The aforementioned application is hereby incorporated by reference in its entirety.

### BACKGROUND

Recent years have seen significant developments in hardware and software platforms for utilizing machine learning tools to analyze underlying datasets and generate machine learning predictions based on different multi-modal data representations. For example, conventional systems can utilize multi-modal representation learning techniques that rely on paired samples to learn common representations and utilize machine learning models to generate predictions from multi-modal data. Despite recent advancements, conventional systems continue to experience a variety of technical problems, including accuracy, flexibility, and efficiency of implementing computing devices in generating paired samples, particularly in fields where measurement devices or other experimental processes destroy underlying samples.

### SUMMARY

Embodiments of the present disclosure provide benefits and/or solve one or more of the foregoing or other problems in the art with systems, non-transitory computer-readable media, and methods for generating perturbation classification scores across different modalities and identifying pairs of data samples across the modalities for a multi-modal machine learning model learning process. For example, the disclosed system matches unpaired samples across disparate modalities. For instance, the disclosed systems generate a first set of perturbation classification scores from a first data modality and generate a second set of perturbation classification scores from a second data modality. Specifically, the disclosed systems compare the first set of perturbation classification scores with the second set of perturbation classification scores to determine a plurality of similarity measures. Further, in some embodiments, the disclosed systems match data samples across the first data modality and the second data modality based on the plurality of similarity scores for a multi-modal machine learning model learning process.

Additional features and advantages of one or more embodiments of the present disclosure are outlined in the description which follows, and in part will be obvious from the description, or may be learned by the practice of such example embodiments.

### BRIEF DESCRIPTION OF THE DRAWINGS

The detailed description provides one or more embodiments with additional specificity and detail through the use of the accompanying drawings, as briefly described below.

FIG. 1A illustrates an overview diagram of the multi-modal pairing system generating similarity measures between disparate modalities for a multi-modal machine learning model learning process in accordance with one or more embodiments.

FIG. 1B illustrate the multi-modal pairing system pairing data samples across disparate modalities in accordance with one or more embodiments.

FIGS. 2A-2B illustrates an example diagram of the multi-modal pairing system training a first classification model and training a second classification model in accordance with one or more embodiments.

FIG. 3 illustrates an example diagram of the multi-modal pairing system applying a perturbation to a biological sample and generating a plurality of similarity measures in accordance with one or more embodiments.

FIG. 4 illustrates an example diagram of the multi-modal pairing system identifying pairs of data samples utilizing a matching algorithm in accordance with one or more embodiments.

FIG. 5 illustrates an example diagram of the multi-modal system matching data samples utilizing an optimal transport matching algorithm in accordance with one or more embodiments.

FIG. 6 illustrates an example diagram of the multi-modal pairing system training a multi-modal machine learning model in accordance with one or more embodiments.

FIG. 7 illustrates an example diagram of the multi-modal pairing system at inference time generating a modality prediction from a disparate modality input in accordance with one or more embodiments.

FIG. 8 illustrates experimental results in accordance with one or more embodiments.

FIG. 9 illustrates additional experimental results of matching disparate modality samples in accordance with one or more embodiments.

FIG. 10 illustrates experimental results of the performance of optimal transport matching on CITE-seq data in accordance with one or more embodiments.

FIG. 11 illustrates experimental results of using a two-sample approach for learning parameters of the multi-modal machine learning model in accordance with one or more embodiments.

FIG. 12 illustrates an example environment of the multi-modal pairing system in accordance with one or more embodiments.

FIG. 13 illustrates an example series of acts to identify pairs of data samples across disparate modalities for a multi-modal machine learning model learning process in accordance with one or more embodiments.

FIG. 14 illustrates a block diagram of a computing device for implementing one or more embodiments.

### DETAILED DESCRIPTION

Embodiments of the present disclosure provide benefits and/or solve one or more of the foregoing or other problems in the art with systems, non-transitory computer-readable media, and methods of a framework for a multi-modal pairing system that matches unpaired multi-modal data for initiating a multi-modal machine learning model learning process. For example, the multi-modal pairing system trains a multi-modal machine learning model to process and translate between disparate modalities (e.g., phenomic image data and protein expression data, or vice-versa) by utilizing paired data samples across the disparate modalities to learn the multi-modal parameters. For instance, in biological applications, underlying data samples are often unpaired (e.g., because testing destroys the underlying cell in individual experiments), which introduces a variety of potentially confounding variables to correctly match across disparate modalities. In some embodiments, the multi-modal pairing system can utilize perturbation classification scores to match unpaired data and train a multi-modal machine learning model. Specifically, the multi-modal pairing system can utilize a first classification model (e.g., a phenomic image classification model) to generate a first set of perturbation classification scores from a first data modality (e.g., phenomic digital images). The multi-modal pairing system can utilize a second classification model (e.g., a protein expression classification model) to generate a second set of perturbation classification scores from a second data modality (e.g., protein expression data). The multi-modal pairing system can compare the first set of perturbation classification scores and the second set of perturbation classification scores to determine similarity measures between pairs of data samples across modalities. The multi-modal pairing system can then train a multi-modal machine learning model based on the matched data samples.

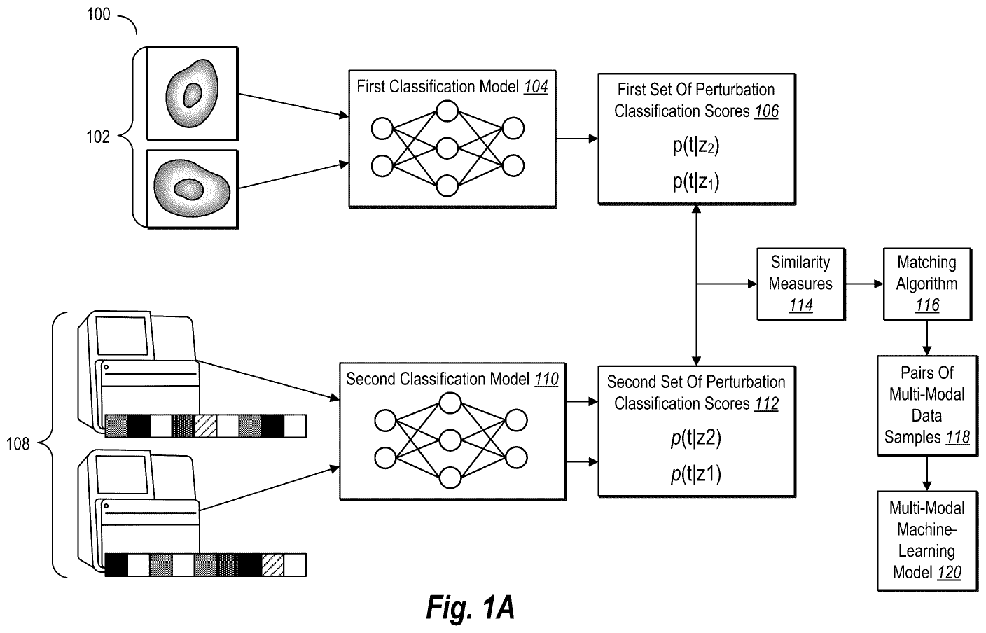

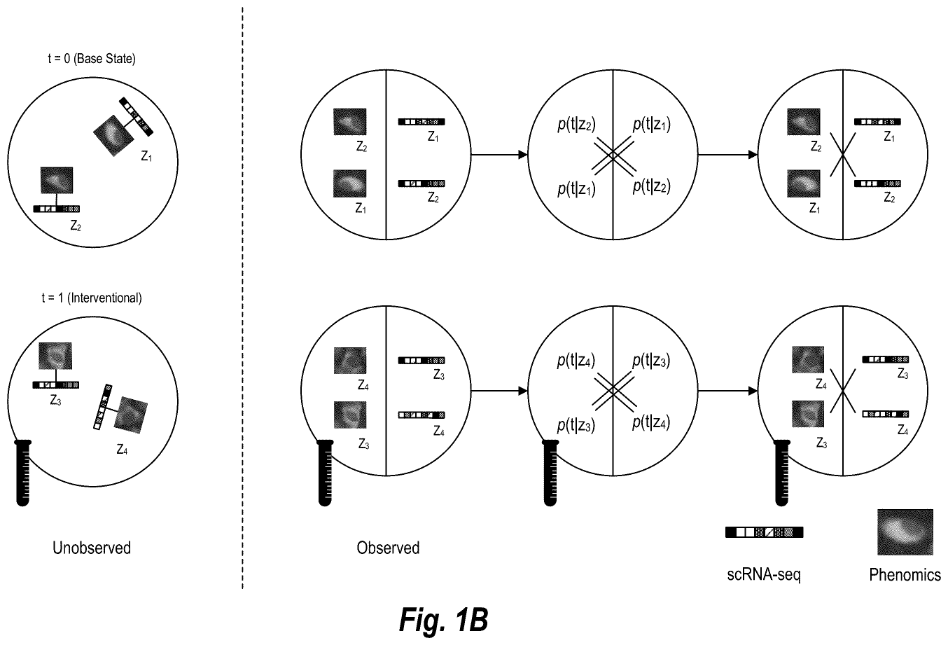

As shown in FIG. 1A, a multi-modal pairing system 100 generates similarity measures for data samples across a first and second modality to learn parameters of a multi-modal machine learning model. For example, as shown, the multi-modal pairing system 100 utilizes classification models to generate classification scores for different modalities. As used herein, the term “classification model” refers to a computer-implemented model that generates a class or category prediction (e.g., a classification for a perturbation applied to a cell). For example, the multi-modal pairing system 100 applies a perturbation to a cell (e.g., applies a drug or performs a CRISPR gene knock out) and generates a prediction or a classification score for one or more perturbations applied to the cell. Specifically, based on input data (e.g., a phenomic digital image or transcriptome data), the multi-modal pairing system 100 utilizes a classification model to generate a classification prediction of the most likely perturbation/intervention applied to a cell.

As shown in FIG. 1A, the multi-modal pairing system 100 utilizes a first classification model 104 to process a specific data modality. As used herein, a “first classification model” or “second classification model” refer to different models trained to analyze features of different data modalities. For example, the multi-modal pairing system 100 can train one classification model on a set of phenomic digital images. For example, the multi-modal pairing system 100 performs imaging on a cell after a perturbation is applied to the cell. Specifically, the multi-modal pairing system 100 utilizes the first classification model 104 to process a phenomic digital image and to further generate a prediction/classification score related to a perturbation applied to the cell.

As used herein, the term “data modality” refers to a particular type or form of data that the multi-modal pairing system 100 collects, processes, and analyzes. For example, the term data modality refers to data collected from different biological features, assays, or representations. Thus, for example, a first data modality can include phenomic digital images a second data modality can include transcriptomic representations (e.g., counts or representations of expression of different transcription proteins), a third data modality can include invivomic data (e.g., data collected from intelligent cages where animals are exposed to different treatments, such as video movement data, temperature data, etc.). Thus, different the data modality includes text, image, video, and/or sensor data (e.g., temperature, pressure, location, etc.) in a variety of different structures.

For instance, the multi-modal pairing system 100 utilizes the first classification model 104 for a first data modality 102 that includes phenomic digital images. As used herein, first data modality, second modality, etc. refers to instances of different data modalities. For example, in relation to FIG. 1, the first data modality 102 refers to phenomic digital images. For instance, the multi-modal pairing system 100 creates the first data modality 102 by exposing cells to a perturbation, developing the cells, and utilizing imaging devices to capture digital images of the cells exposed to the perturbation (e.g., the phenomic digital images). Specifically, the multi-modal pairing system 100 uses the first classification model 104 to process data from the first data modality 102 to generate predictions related to the first data modality (e.g., which perturbation were applied to the cell).

Moreover, as shown, the multi-modal pairing system 100 utilizes the first classification model 104 to generate a first set of perturbation classification scores 106. As used herein, a set perturbation classification scores refers to a plurality of predictions/scores generated by a classification model. For example, the multi-modal pairing system 100 utilizes the first classification model 104 to generate the first set of perturbation classification scores 106 from a plurality of phenomic digital images. Specifically, each of the classification scores of the first set of perturbation classification scores 106 includes a probability distribution of a phenomic digital image. In other words, a classification score assigns a level of confidence or probability of a phenomic digital image having a specific perturbation applied to a cell. To illustrate, the human genome contains 17,000 genes and the multi-modal pairing system 100 generates a probability distribution for each perturbation (e.g., of the 17,000 genes or each of a plurality of compounds). The highest probability of a perturbation classification score can thus correspond to a specific perturbation/gene knockout (e.g., gene 125).

As also shown in FIG. 1A, the multi-modal pairing system 100 utilizes a second classification model 110 to process a second data modality 108. As discussed above, a second classification model refers to a different classification model than a first classification model (e.g., trained to analyze protein expression data of a cell. To illustrate, a second classification model can analyze cell expression data and predict a perturbation applied to the cell. For example, the multi-modal pairing system 100 processes biological data such as cells with a machine to generate a representation of transcript proteins (e.g., mRNA/RNA sequencing data) and further utilizes the second classification model 110 to process this transcriptomic representation. From processing this transcriptomic representation, the multi-modal pairing system 100 generates predictions for perturbations applied to the cell.

As mentioned previously, a second data modality refers to a different data modality (e.g., protein expression data). For example, the second data modality 108 refers to RNA sequencing data. For instance, the multi-modal pairing system 100 creates the second data modality 108 by exposing cells to a perturbation and further utilizing a transcription machine to generate a transcriptomic representation of the cell. Specifically, the multi-modal pairing system 100 uses the second classification model 110 to process the protein expression data (e.g., RNA sequencing data) to generate predictions related to the second data modality 108 (e.g., which perturbations were applied to the cell).

As shown, the multi-modal pairing system 100 utilizes the second classification model 110 to generate a second set of perturbation classification scores 112. As mentioned, a second set of perturbation classification scores includes a different set of predictions/scores generated by the second classification model 110. For example, the multi-modal pairing system 100 utilizes the second classification model 110 to generate the second set of perturbation classification scores 112 from protein expression data. Specifically, each of the classification scores of the second set of perturbation classification scores 112 includes a probability distribution of protein expression data. In other words, a classification score assigns a level of confidence or probability of a protein expression sequence (e.g., a RNA sequence) having a specific perturbation applied to a cell.

Furthermore, the multi-modal pairing system 100 can generate/determine similarity measures 114. As used herein, the term “similarity measure” refers to a quantitative metric that indicates a degree of closeness, relatedness, or correspondence between elements. For example, the term similarity measure refers to a degree of how similar a sample or element from one modality is to another sample or element from another modality. For example, the multi-modal pairing system 100 compares the first set of perturbation classification scores 106 with the second set of perturbation classification scores 112 to determine a plurality of similarity measures 114.

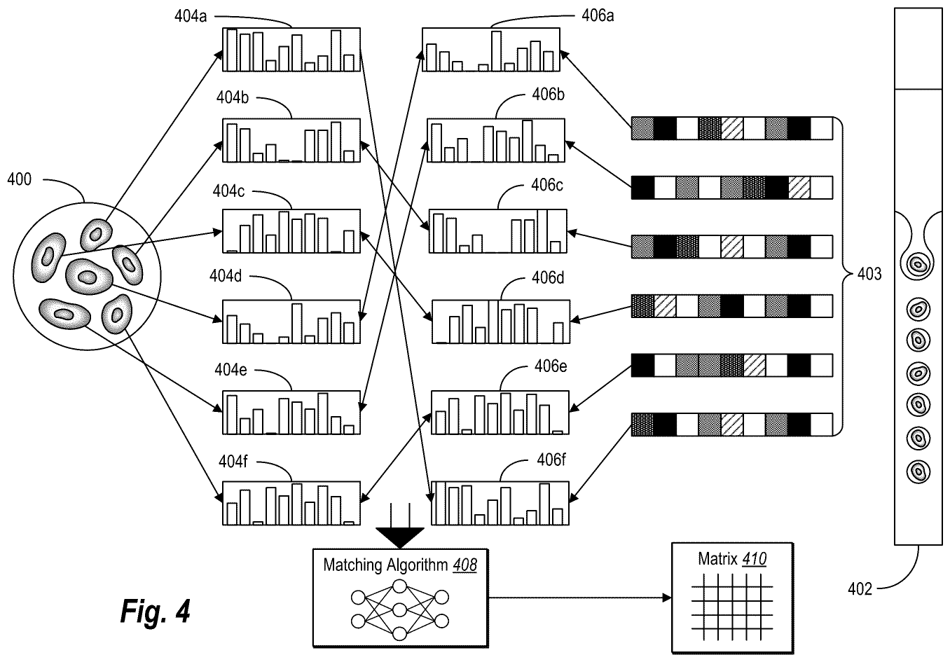

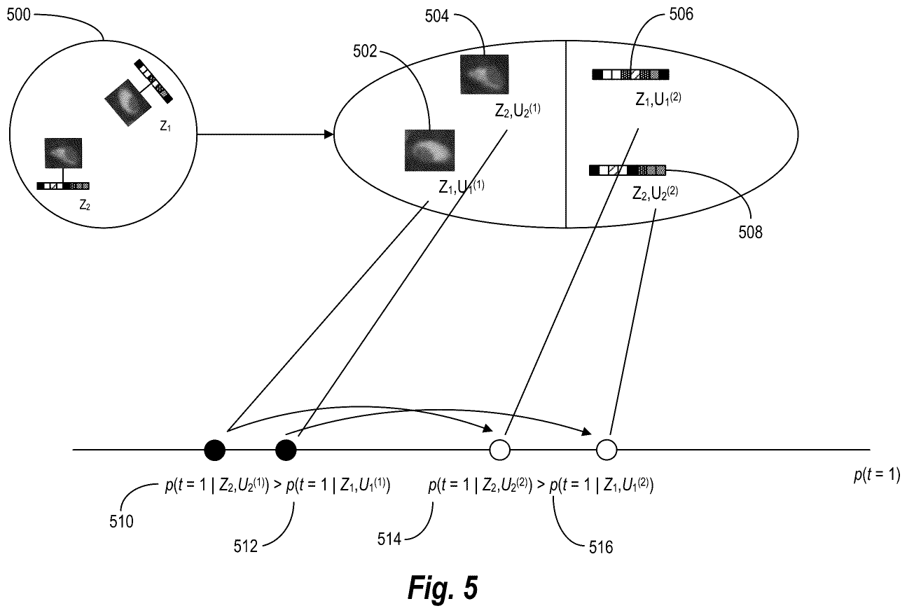

As further shown, the multi-modal pairing system 100 utilizes a matching algorithm 116 to match unpaired data samples across the first data modality 102 and the second data modality 108. Additional details regarding the matching algorithm 116 is given below in the description of FIGS. 4 and 5. As shown, from the matching algorithm 116, the multi-modal pairing system 100 identifies pairs of multi-modal data samples 118. As used herein, the term “pairs of data samples” refers to samples or elements matched across modalities. For example, the term “pairs of data samples” refers to the multi-modal pairing system 100 utilizing the plurality of similarity measures 114 to match various pairs of data samples from the first data modality 102 and the second data modality 108. Specifically, a data sample pair can include a sample or element from the first data modality 102 that is closest in similarity to another sample or element in the second data modality 108. Moreover, the multi-modal pairing system 100 can use the pairs of data samples to initiate the multi-modal machine learning model learning process because the pairs of data samples can maximize the shared information between disparate data modalities.

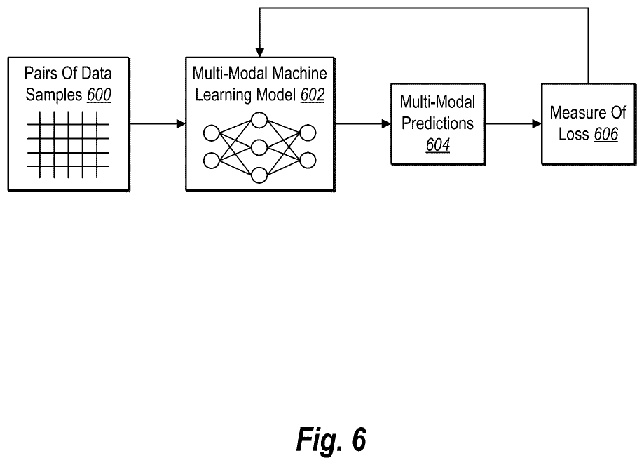

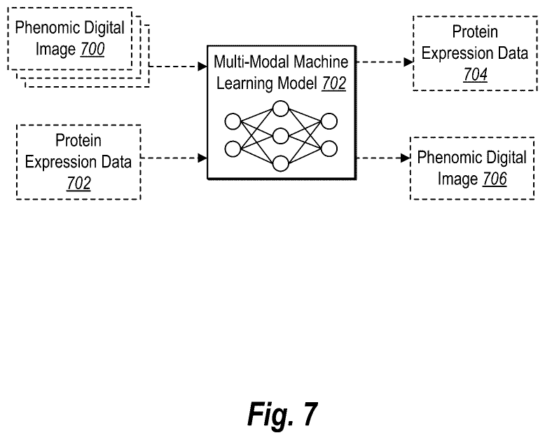

As also shown in FIG. 1A, the multi-modal pairing system 100 provides the pairs of multi-modal data samples 118 to a multi-modal machine learning model 120. For example, the multi-modal pairing system 100 provides the multi-modal data samples 118 to initiate a multi-modal machine learning model learning process, which is discussed in additional detail below in the description of FIGS. 6 and 7.

As mentioned above, conventional systems suffer from a number of technical deficiencies that can be addressed by the multi-modal pairing system 100. For example, conventional systems suffer from operational inflexibility because they cannot efficiently generate or identify training data across modalities. Specifically, conventional systems depend on the availability of already paired samples (e.g., paired image and text captions) across data modalities. For instance, conventional systems typically need already paired samples to explicitly learn representations to maximize matching between different modes (e.g., image and text). In conventional systems, paired image-text captioning data can be much more accessible than trying to obtain paired data in the tech-bio space. For example, conventional systems in the tech-bio space attempt to collect experimental data and perform experimental analysis on biological samples, however the collection and analysis of data often results in the destruction of underlying samples.

Specifically, in the tech-bio space, conventional systems typically only have access to unpaired data due technical reasons. For instance, RNA sequencing, protein expression assays, and the collection of microscopy images for experimental assays can kill the cell to take measurements. Because of this, conventional systems are often unable to collect multiple different measurements from the same cell and thus only explicitly group cells by experimental conditions. As a result, conventional systems are operationally inflexible because they rely on already paired data and specifically, they are unable to obtain multi-modal pairs in the tech-bio space.

In addition, conventional systems are also inaccurate. In particular, without cross-modality training pairs conventional systems cannot train and develop accurate multi-modal machine learning models. For example, observations from disparate data modalities typically exist in entirely different spaces. For instance, microscopy images of cells are in the pixel space while gene expression data is in the form of mRNA abundance counts. Moreover, even if measurements or observations could be performed in the same space, determining an accurate way to match unpaired samples remains a non-trivial task for conventional systems (e.g., conventional systems struggle to find metrics for matching unpaired samples that are biologically relevant). In other words, conventional systems cannot teach models to generate multi-modal machine learning representations that accurately embed and reflect underlying latent features of cross-modality samples.

Conventional systems that do attempt to train multi-modal machine learning models are extremely inefficient and require excessive time and computer resources. Indeed, to generate a training dataset, conventional systems would require extensive duplication of complex robotic experiments to attempt to generate training pairs across different modalities. Accordingly, conventional systems require significant computer resources of implementing systems that are still plagued by the problems discussed above.

The multi-modal pairing system 100 provides a variety of technical benefits and address technical problems of conventional systems. For example, the multi-modal pairing system 100 can improve operational flexibility of implementing computing devices that do not depend on the availability of already paired samples across data modalities. Specifically, the multi-modal pairing system 100 matches unpaired data samples across disparate modalities by utilizing a first classification model and a second classification model to generate respective perturbation classification scores for each data modality. Indeed, by training multiple classifiers and utilizing the classifiers to generate perturbation classification scores for unpaired multi-modal data, the multi-modal pairing system 100 can generate a plurality of similarity measures (e.g., cross-modality training pairs) between the first data modality and the second data modality. Moreover, the multi-modal pairing system 100 can utilize the plurality of similarity measures to identify pairs of data samples for training multi-modal machine learning models to generate multi-modal learning representations.

Accordingly, in the tech-bio space, the multi-modal pairing system 100 overcomes issues related to the destruction of underlying biological samples, thus increasing the operational flexibility relative to conventional systems. Specifically, for RNA sequencing, protein expression assays, and the collection of microscopy images for experimental assays, the multi-modal pairing system 100 matches unpaired data from multiple different measurements from different cells by training multiple classifiers to generate perturbation classification scores for their respective data modality. From the respective perturbation classification scores, the multi-modal pairing system 100 further matches the unpaired data samples and utilizes the pairs of data samples to initiate a multi-modal machine learning model learning process.

Moreover, in some embodiments, the multi-modal pairing system also improves accuracy relative to conventional systems. In contrast to conventional systems (e.g., which struggle with data modalities existing in entirely different spaces and finding a way to match unpaired samples in a biologically relevant manner), the multi-modal pairing system 100 accurately matches unpaired data samples using separately trained classifiers by comparing perturbation classification scores across disparate data modalities to determine similarity measures. Indeed, the multi-modal pairing system 100 can then utilize these similarity measures (e.g., cross-modality training pairs) to build multi-modal machine learning models that generate accurate embeddings for comparison within a common feature space.

Furthermore, the multi-modal pairing system 100 also improves efficiency relative to conventional systems. By generating pairs of data samples based on the plurality of similarity measures, the multi-modal pairing system 100 can transform existing data collections into matched training data for building multi-modal machine learning models. Indeed, the multi-modal pairing system can avoid the significant time and resources required to collect matched pairs of training data by analyzing existing multi-modal datasets and generating cross-modality pairs for training multi-modal machine learning models.

FIG. 1A illustrates a general overview of the multi-modal pairing system 100 generating pairs of multi-modal data samples 118. FIG. 1B provides additional details regarding theoretical assumptions of the multi-modal pairing system 100 matching unpaired data samples across disparate data modalities.

As mentioned above, multi-modal representation learning techniques can rely on paired samples to learn common representations, but paired samples are challenging to collect in fields such as biology where measurement devices often destroy the data samples. The multi-modal pairing system 100 can address the challenge of matching unpaired samples across disparate modalities in multi-modal representation learning. In one or more embodiments, this approach of matching unpaired samples draws an analogy between potential outcomes (e.g., perturbation or no perturbation) in causal inference (e.g., a cause-and-effect relationship) and potential views in multi-modal observations (e.g., views such as a phenomic digital image or protein expression data), which allows the multi-modal pairing system 100 to utilize Rubin's framework to estimate a common space in which to match data samples from disparate modalities. See Rubin, D. B., Estimating causal effects of treatments in randomized and nonrandomized studies. Journal of educational Psychology, 66 (5): 688, 1974 (hereinafter “Rubin”), which is incorporated by reference in its entirety herein.

In particular, the multi-modal pairing system 100 can operate with an assumption that data samples are collected that are experimentally perturbed by treatments. From this, the multi-modal pairing system 100 estimates a similarity score from each modality, which encapsulates shared information between a latent state and perturbation treatment and can be used to define a distance between data samples of different modalities.

To understand the matching problem more abstractly, consider multiple modalities as potential “views”, x⁽¹⁾(z) ∈ [symbol] ⁽¹⁾, x⁽²⁾(z) ∈ [symbol] ⁽²⁾ of the same underlying latent state, z∈ [symbol] , where one view is observed for any individual unit, i (e.g. for an individual cell, one view can be a phenomic digital image or RNA sequencing data). If z were observed for each sample, this would address the issue of different modalities existing in entirely different spaces, by providing a common space, Z, to match samples. But for observations of complex systems like those observed in biological samples, the latent state shared between different modalities faces the challenge of not knowing what the “right” metric is for measuring similarity between samples in the latent space.

For instance, the multi-modal pairing system 100 leverages a key observation that potential views of a sample, x⁽¹⁾(z), x⁽²⁾(z), are analogous to potential outcomes in Rubin's framework in causal inference. Accordingly, either the outcome, Y(t=1), of applying a perturbation treatment, t=1 to a cell, or the outcome, Y(t=0), of a placebo (e.g., no perturbation treatment is applied), t=0, is available. Consequently, to match disparate modalities based on biologically relevant data, the multi-modal pairing system 100 shifts the focus to matching on the subset of the latent space Z, that is directly affected by the experimental perturbations, denoted as t. Given this connection with Rubin's framework, the multi-modal pairing system 100 can leverage the similarity score, τ⁽ⁱ⁾(x):=P(t|X⁽ⁱ⁾), which represents the coarsest transformation of the latent space Z (e.g., a lower-dimensional space that retains useful information with reduced complexity) that still captures all the information about the perturbation T, in the sense that after conditioning on the similarity score, τ(Z), any remaining variation in Z is independent of the perturbation T. Thus, the similarity score defines a space for matching that is shared across disparate modalities, and that is sensitive to the variation in Z that results from experimental perturbations, T.

As demonstrated below, the similarity score captures all the shared information between the latent state, Z, and the perturbation treatment T, and importantly, if each map from latent state to the respective modalities (e.g., phenomic digital image or protein expression data), denoted as X^{(e)}, is injective (e.g., is a one-to-one function, where each input corresponds to a unique output), then the multi-modal pairing system 100 can learn to make predictions in a modality from another modality, and still capture all of this shared information. As mentioned above, in practice, to estimate a similarity score the multi-modal pairing system 100 trains two classifiers—one for each modality—to predict which treatment was applied to the respective modalities, and then matches across modalities based on the similarity between predicted perturbation treatments within each treatment group.

The similarity score defines a space for matching, but the multi-modal pairing system 100 also addresses the fact that the same unit does not appear in both modalities. In other words, the multi-modal pairing system 100 does not have the luxury of using the exact same biological sample (e.g., cell) to generate a phenomic digital image as it does to generate a RNA sequence. If every unit appeared in both modalities, the multi-modal pairing system 100 could use nearest neighbor matching (discussed below), but because different units (individual cells) are sampled in the different modalities, the multi-modal pairing system 100 can better estimate the missing modality by combining multiple observations.

For example, FIG. 1B provides an illustration of the multi-modal pairing system 100 generating multi-modal machine learning pairs in accordance with one or more embodiments. For instance, FIG. 1B shows unobserved modalities of a base state (e.g., no perturbation treatment) where phenomic digital images are matched with RNA sequences and an intervention (e.g., a cell with an applied perturbation treatment) where phenomic digital images are also matched with RNA sequences. Furthermore, FIG. 1B shows two observed modalities (e.g., single-cell RNA sequencing (e.g., scRNA-seq) and phenomics) are generated in pairs according to some common latent variable, zᵢ, but the two datasets are observed separately. The multi-modal pairing system 100 can access observations from both modalities from a base unperturbed state and at least one interventional state. The multi-modal pairing system 100 can train separate classifiers for the intervention label to reveal the shared information p(t|zᵢ), which allows the multi-modal pairing system 100 to re-pair the observed disconnected modalities.

Consider multi-modal settings where there exist two potential views, X^{(e)} ∈X^{(e)} from two different modalities indexed by, e ∈{1,2}. The observations will typically be in very different spaces: for example, X⁽¹⁾, may be the space of images of cells under a microscope, and X⁽²⁾ may be the space of gene expression data. The latent state of these observations is experimentally perturbed by some treatment, t, that is observed. This process defines a jointly distributed random variable, (X⁽¹⁾, X⁽²⁾, e, t) from which is observed only a single modality, its index and treatment assignment, {xᵢ^{(e}^{ᵢ}⁾, eᵢ, tᵢ}ᵢ₌₁ⁿ. The multi-modal pairing system 100 matches or estimates the samples from the missing modality (e.g., the multi-modal pairing system 100 predicts a second modality from a first modality) that would correspond to the realization of the missing random variable. In some embodiments, the multi-modal pairing system 100 can match within perturbation treatment groups (e.g., the treatment variable is only used to learn a common space in which to match).

For example, assume each modality arises from a common latent random variable Z representing the underlying process, as follows, in Equation (1):

$${Z}^{( t )} \sim {P}_{z}^{( t )} , {U}^{( e )} \sim {P}_{U}^{( e )} , \text{} {X}^{( e , t )} = {f}^{e} ( {Z}^{( t )} , {u}^{( e )} ) , {u}^{( e )} ⊥ ⊥ Z , {P}_{z}^{( j )} for each i \neq j , t = 1 , ... , T ,$$

where t indexes the experimental perturbations that have a non-trivial effect on the distribution of the latent variables. In these structural equations, f^{e} represents the measurement process that captures the latent state: for example, in a microscopy image, this would be the microscope and camera that maps a cell to pixels. The modality specific noise variables, u⁽¹⁾⊥⊥u⁽²⁾, play the role of measurement noise and modality-specific factors of variation: e.g. u⁽¹⁾, and can describe the layout and orientation of cells on a slide.

Under this model, if Z were observable, an optimal matching can be constructed by exactly matching the modalities with the most similar Z. Because Z is latent, one might hope to first recover Z by fitting a generative model, but the problem of exact recovery is difficult because of theoretical difficulties such as identifiability and disentangling Z from the modality-specific noise terms u^{(e)} In one or more implementations, the multi-modal pairing system 100 matches on the similarity score of Z with respect to t, which is defined as πᵢ(Z):=P(t|Z). The multi-modal pairing system 100 need not observe Z (or compute it directly) but if f^{(e)} is injective (one-to-one mapping) for e=1, 2, then the multi-modal pairing system 100 can compute the similarity score from each of the observed modalities (e.g., since P(t|Z)=P(t|X^{(e)}) if f^{(e)} is injective). Effectively, the interventions, t, provide an observable link between the modalities, thereby revealing information about Z^{(t)}. Not only does it reveal shared information, classical causal inference states that it can capture all shared information, as well as by doing so minimally, in terms of having minimum dimension and entropy. Consider the following proposition:

Proposition A. In the model described by Equation (1), further assume that f^{(e)} are injective for e=1,2. Then, the similarity scores in either modality is equal to the similarity score given by Z^{(t)}, i.e., π(X^{(1,t)})=π(X^{(2,t)})=π(Z^{(t)}) as random variables. This implies Equation (2):

$$I ( t , {Z}^{( t )} \vert π ( {Z}^{( t )} ) ) = I ( t , {Z}^{( t )} \vert π ( {X}^{( t )} ) ) = 0 ,$$

for each t, where I is the mutual information. Furthermore, any other function b(Z^{(t)}) satisfying I(t, Z^{(t)}|b(Z^{(t)}))=0 is such that π(Z^{(t)})=f(b(Z^{(t)})).

Proof. Let x^{(e,t)} denote the observed modality and z^{(t)}, u^{(e)} be the unique corresponding latent values. Injectivity gives Equation (3):

$$\begin{matrix}π ( {x}^{( e , t )} ) = P ( t \vert {X}^{( e , t )} = {x}^{( e , t )} ) \\ = P ( t \vert {Z}^{( t )} = {z}^{( t )} , {U}^{( e )} = {u}^{( e )} ) \\ = P ( t \vert {Z}^{( t )} = {z}^{( t )} ) = π ( {z}^{( t )} ) ,\end{matrix}$$

for e=1,2, given the assumption U^{(e)}⊥⊥t|Z^{(t)} in Equation (1). Since this holds pointwise, it shows that π(X^{(1,t)})=π(X^{(2,t)})=π(X^{(t)})=π(Z^{(t)}) as random variables. Now, a classical result of Rubin gives that Z^{(t)}⊥⊥t|π(Z^{(t)}), and that for any other function b (a balancing score) such that (Z^{(t)})=g(b(Z^{(t)})). The first property written in information theoretic terms yields, Equation (4):

$$I ( t , {Z}^{( t )} \vert π ( {Z}^{( t )} ) ) = I ( t , {Z}^{( t )} \vert π ( {X}^{( t )} ) ) = 0 ,$$

since π(X^{(t)})=π(Z^{(t)}) as random variables.

The above shows that computing the similarity score on either modality is equivalent to computing it on the unobserved shared latent, which captures all the shared information observable in t. The final statement implies that it is of minimal dimension and entropy, and thus it discards the modality-specific information that may be counterproductive to matching.

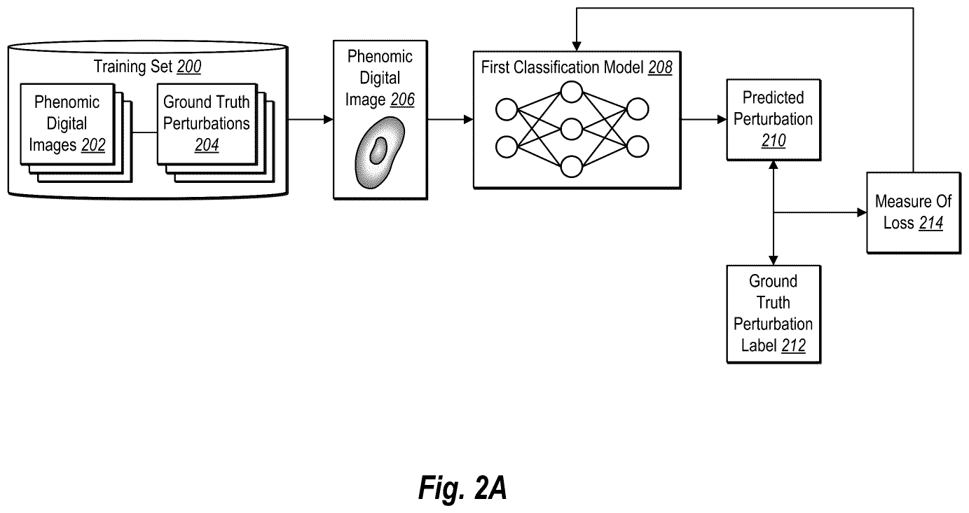

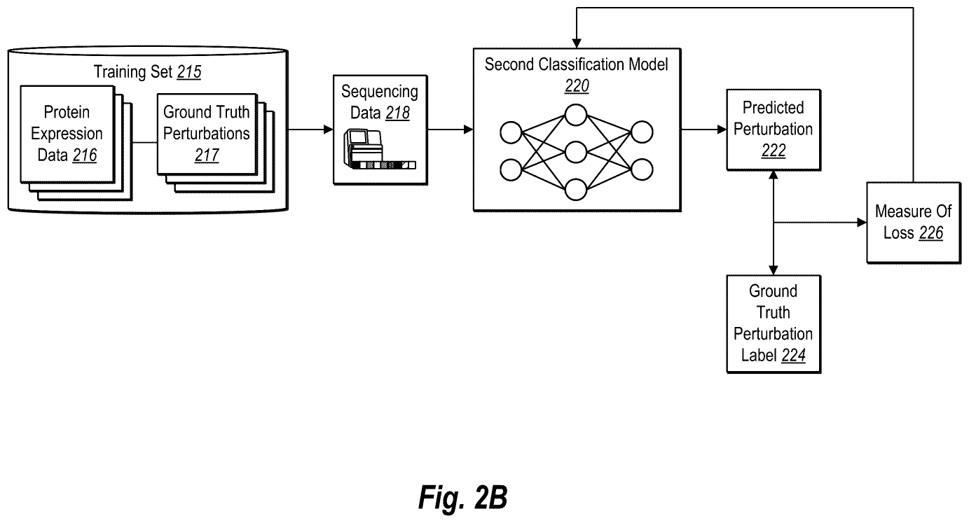

As mentioned above, the multi-modal pairing system 100 separately trains classifiers to generate predictions for disparate data modalities. As shown, FIG. 2A illustrates the multi-modal pairing system 100 training a first classification model for a first modality in accordance with one or more embodiments. The multi-modal pairing system 100 can train separate machine learning classifiers (e.g., encoders with linear classification heads) utilizing these datasets reflecting individual modalities. For example, the multi-modal pairing system 100 can utilize a dataset of a first modality (e.g., phenomic digital images) to train a first machine learning classifier (e.g., a convolutional neural network) to analyze features of a phenomic digital image of a cell and predict the treatment perturbation applied to the cell.

As shown, the multi-modal pairing system 100 receives a training set 200 that includes phenomic digital images 202 and ground truth perturbations 204. As used herein, the term “training set” refers to a set of data with input data and corresponding output labels or classifications. Specifically, the multi-modal pairing system 100 utilizes the training set 200 to learn parameters of a classification model.

For example, as mentioned, the multi-modal pairing system 100 can collect multi-modal data reflecting experimental results in different modalities from treatments of underlying samples. To illustrate, the multi-modal pairing system 100 can generate a first dataset to train the first classification model 208 that reflects a first modality (e.g., phenomic digital images). To illustrate, the multi-modal pairing system 100 can apply various perturbation treatments (e.g., CRISPR-gene knockouts or compound treatments) to cells, develop the cells, and then capture phenomic digital images that portray the cells resulting from these perturbations. Additionally, the term “ground truth perturbations” refers to a label or classification corresponding with a phenomic digital image. Specifically, a ground truth perturbation can indicate a specific perturbation applied to a cell as captured in a phenomic digital image.

As shown in FIG. 2A, the multi-modal pairing system 100 utilizes a first classification model 208 to process a phenomic digital image 206 to generate a predicted perturbation 210 (e.g., a prediction of a specific perturbation shown in the phenomic digital image 206) and compares the predicted perturbation with a ground truth perturbation label 212. From the comparison, the multi-modal pairing system 100 determines a measure of loss 214 and modifies parameters of the first classification model 208.

As shown in FIG. 2B, the multi-modal pairing system 100 trains a second classification model for a data modality different than the modality in FIG. 2A in accordance with one or more embodiments. For example, the multi-modal pairing system 100 can utilize a dataset of a second modality (e.g., scRNA-seq) to train a second machine learning model classifier (e.g., a fully connected multi-layer perceptron) to analyze protein data (e.g., different RNA counts/expressions) and predict the treatment perturbation.

Similar to FIG. 2A, FIG. 2B shows the multi-modal pairing system 100 receiving a training set 215 that includes protein expression data 216 and ground truth perturbations 217. For example, the multi-modal pairing system 100 can generate a second dataset that reflects a second modality (e.g., protein expression) to train a second classification model 220. To illustrate, the multi-modal pairing system 100 can apply various perturbation treatments to cells and then utilize sequencing machines to analyze (e.g., count or otherwise measure) the resulting expression of proteins/RNA within the cells.

As shown in FIG. 2B, the multi-modal pairing system 100 utilizes the second classification model 220 to process sequencing data 218 to further generate a predicted perturbation 222 (e.g., predict a perturbation applied to a cell that results in the sequencing data 218). Further, as shown, the multi-modal pairing system 100 compares the predicted perturbation 222 with a ground truth perturbation label 224 to determine a measure of loss 226 to modify parameters of the second classification model 220.

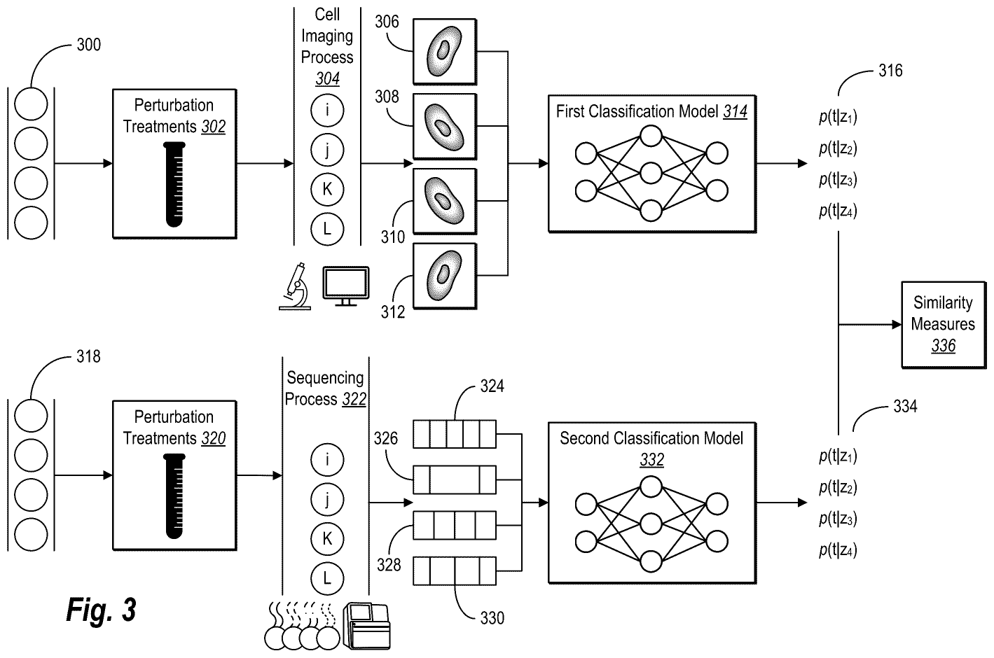

As mentioned above, the multi-modal pairing system 100 applies perturbation treatments to biological samples and further generates similarity measures. As shown in FIG. 3, the multi-modal pairing system 100 generates perturbation classification scores and compares the perturbation classification scores across disparate modalities to determine the similarity measures in accordance with one or more embodiments.

As shown, the multi-modal pairing system 100 receives a biological sample 300 and a biological sample 318. Specifically, the biological samples 300 and 318 can include a cell (or a collection of cells). As used herein, the term “cell” refers to a structural, functional, and biological unit of living organisms. Specifically, a cell can vary in size, shape, and function depending on the organism and the role of the cell. For example, a cell can include a plasma membrane to separate the internal cell environment from the external surroundings and the cell can further contain genetic material.

As shown, the multi-modal pairing system 100 applies perturbation treatments 302 to the biological sample 300 and perturbation treatments 320 to biological sample 318. As used herein, the term “perturbation” (e.g., cell perturbation) refers to an alteration or disruption to a cell or the cell's environment (to elicit potential phenotypic changes to the cell). In particular, the term perturbation can include a gene perturbation (i.e., a gene-knockout perturbation) or a compound perturbation (e.g., a molecule perturbation or a soluble factor perturbation). These perturbations are accomplished by performing a perturbation experiment. A “perturbation experiment” refers to a process for applying a perturbation to a cell. A perturbation experiment also includes a process for developing/growing the perturbed cell into a resulting phenotype.

In some instances, the multi-modal pairing system 100 applies an individual perturbation to a biological sample. As used herein, the term “individual perturbation” refers to the multi-modal pairing system 100 exposing a cell to a single perturbation. Specifically, the multi-modal pairing system 100 performs a perturbation experiment that involves an individual perturbation to the cell. For example, the multi-modal pairing system 100 performs a single gene knockout on the cell.

As shown, the multi-modal pairing system 100 performs a cell imaging process 304 on the biological sample 300 with the perturbation treatments 302 applied. As used herein, the term “cell imaging process” refers to a technique to visualize cells utilizing microscopes. As shown, the multi-modal pairing system 100 utilizes the cell imaging process 304 to generate phenomic digital images 306-312. As used herein, the term “phenomic digital images” refers to a digital image portraying a cell (e.g., a cell after applying a perturbation). For example, a perturbation digital image includes a digital image of a stem cell after application of a perturbation and further development of the cell. Thus, a perturbation digital image comprises pixels that portray a modified cell phenotype resulting from a particular cell perturbation.

Further, as shown, the multi-modal pairing system 100 utilizes a first classification model 314 to process the phenomic digital images 306-312. As mentioned above, the first classification model 314 includes a phenomic image classification model to classify/generate predictions from phenomic digital images. Specifically, the multi-modal pairing system 100 utilizes the phenomic image classification model to process images of perturbed cells to generate classification scores.

Moreover, from processing the phenomic digital images 306-312, the multi-modal pairing system 100 generates a first set of perturbation classification scores 316. As mentioned above, the first set of perturbation classification scores includes a probability distribution of a phenomic digital image. In other words, a classification score assigns a level of confidence or probability of a phenomic digital image having a specific perturbation applied to a cell.

As shown in FIG. 3, the multi-modal pairing system 100 also utilizes a sequencing process 322 to process the biological sample 318 with the perturbation treatments 320. As shown in FIG. 3, the multi-modal pairing system 100 utilizes the sequencing process 322 to generate protein expression data 324-330. Specifically, the multi-modal pairing system 100 can utilize a transcription machine to sequence the biological sample 318 with the perturbation treatments 320.

As used herein, the term “transcription machine” refers to a machine for transcribing protein expression data (e.g., RNA sequences). For example, the multi-modal pairing system 100 utilizes the transcription machine to convert RNA into complementary DNA (cDNA) and sequencing the cDNA to determine the RNA sequences present in the biological sample. For instance, the multi-modal pairing system 100 collects the biological sample that includes the RNA and extracts from the sample (e.g., removing the protein, DNA, and other cellular components). Further, the multi-modal pairing system 100 transcribes the RNA into cDNA, fragments the cDNA and sequences the cDNA.

As used herein, the term “protein expression data” refers to information obtained from the measurement of protein levels within a biological sample (e.g., a cell or tissue). For example, protein expression data can include a relative level/number of protein within a biological sample, types of proteins expressed in a biological sample, and protein expression over time in a biological sample. Furthermore, the protein expression data includes RNA sequencing data. For instance, RNA sequencing data includes data that indicates which genes in a biological sample (e.g., a cell) is being actively transcribed. Moreover, RNA sequencing data also can indicate variations in gene expression (e.g., due to a perturbation).

As further shown, the multi-modal pairing system 100 utilizes a second classification model 332 to process the protein expression data 324-330. As mentioned above, the second classification model 332 includes a protein expression classification model. For example, the protein expression classification model can generate a prediction for a perturbation applied to a cell based on a protein expression measurement of a cell (e.g., a cell exposed to the perturbation treatments 320).

Moreover, the multi-modal pairing system 100 generates a second set of perturbation classification scores 334. As mentioned above, the second set of perturbation classification scores includes a probability distribution of a protein expression data. In other words, a classification score assigns a level of confidence or probability of the protein expression data having a specific perturbation applied to a cell.

As shown, the multi-modal pairing system 100 compares the first set of perturbation classification scores 316 with the second set of perturbation classification scores 334 to determine similarity measures 336. Specifically, each similarity measure indicates a level of how similar a phenomic digital image is to protein expression data. For instance, the multi-modal pairing system 100 can generate similarity measures of a phenomic digital image to each individual protein expression data (e.g., each RNA sequence).

In some embodiments, the multi-modal pairing system 100 can map/project the first set of perturbation classification scores 316 and the second set of perturbation classification scores 334 to a single feature space. Specifically, the multi-modal pairing system 100 generates a matrix of the unpaired data samples which includes a probability indicating a similarity between the unpaired data samples. Furthermore, the multi-modal pairing system 100 can use the matrix as a representation of each modality in the shared feature space.

FIG. 4 provides additional details of matching unpaired data samples across disparate data modalities in accordance with one or more embodiments. As mentioned, upon training, the multi-modal pairing system 100 can then utilize these classification models (e.g., machine learning models) to generate similarity scores and ultimately predict matches between cross-modality samples.

As shown, the multi-modal pairing system 100 performs a cell imaging process on a biological sample 400 with a perturbation treatment. For example, the multi-modal pairing system 100 can utilize a first classifier to generate a first perturbation classification score for a treatment from a first sample (reflecting the first modality of phenomic digital images). As discussed above, the multi-modal pairing system 100 generates a first set of perturbation classification scores 404a-404f.

As mentioned, the multi-modal pairing system 100 utilizes biological sample 402 with applied perturbations to determine protein expression data 403. From the protein expression data 403, the multi-modal pairing system 100 generates a second set of perturbation classification scores 406a-406f for the treatment from a second sample (reflecting the second modality of protein expression data).

As illustrated in FIG. 4, the first set of perturbation classification scores 404a-404f and the second set of perturbation classification scores 406a-406f show probability distributions. Specifically, the probability distribution indicates a probability or confidence level of each cell and the likelihood of an applied perturbation. For instance, a cell can contain a high likelihood of first, second, and third perturbation being applied to it, but a low likelihood of a fourth or fifth perturbation being applied to it. Accordingly, the trained classifiers can generate a probability distribution for each cell for the likelihood of the cell having an applied perturbation.

Upon generating these perturbation classification scores, the multi-modal pairing system 100 can compare the perturbation classification scores (e.g., determine a cross-modality distance measurement) to identify matches based on the similarity measures between the disparate modalities. In particular, the multi-modal pairing system 100 can utilize a matching algorithm 408 (e.g., shared nearest neighbors matching, optimal transport matching, or other matching algorithms) to identify pairs of data samples (e.g., cross-modality sample matches or cross-modality pairs).

As used herein, the term “matching algorithm” refers to a model or an algorithm that identifies pairs of data samples. For example, the matching algorithm 408 can include shared nearest neighbors matching or optimal transport matching. Specifically, the multi-modal pairing system 100 analyzes the first set of perturbation classification scores 404a-404f and the second set of perturbation classification scores 406a-406f and identifies pairs of data samples across the first data modality and the second data modality that are most similar to each other.

As shown, from identifying the pairs of data samples, the multi-modal pairing system 100 further generates a matrix 410. As used herein, a “matrix” refers to a two-dimensional array arranged in rows and columns. Specifically, the matrix 410 includes rows with a set of phenomic digital images and columns with protein expression data (e.g. RNA sequences) or vice-versa. Moreover, the matrix 410 includes entries corresponding to a specific phenomic digital image-protein expression data pair. For example, a specific entry of the matrix 410 can indicate a probability of an element from the second data modality matching an element from the first data modality.

Given a multi-modal dataset with observations {xᵢ⁽¹⁾, 1, tᵢ}ᵢ₌₁ⁿ^{₁} and {xⱼ⁽²⁾, 2, tⱼ}ⱼ₌₁ⁿ^{₂}, the multi-modal pairing system 100 can compute a matching matrix (or coupling) between the two modalities. In the above notation for the multi-modal dataset, xᵢ⁽¹⁾ represents a first data sample from the first data modality, xⱼ⁽²⁾ represents a second data sample from the second data modality, 1 and 2 represent an index (e.g., a reference to a row or column within a matrix), and tᵢ and tⱼ represents a perturbation treatment assignment.

The multi-modal pairing system 100 can define a n₁×n₂ matching matrix M where Mᵢⱼ represents the likelihood of xᵢ⁽¹⁾ being matched to xⱼ⁽²⁾. The multi-modal pairing system 100 can normalize ΣⱼMᵢⱼ=1, (but does not necessarily formalize the entries of Mᵢⱼ as probabilities otherwise). In some implementations, to reduce confusion, the multi-modal pairing system 100 can index observations in modality 1 by i and observations in modality 2 by j. In one or more implementations, the multi-modal pairing system 100 can perform matching within observations with the same value of t, to obtain a matrix Mₜ for each t. In other words, the multi-modal pairing system 100 can generate a matrix for each type of perturbation treatment that matches across disparate modalities.

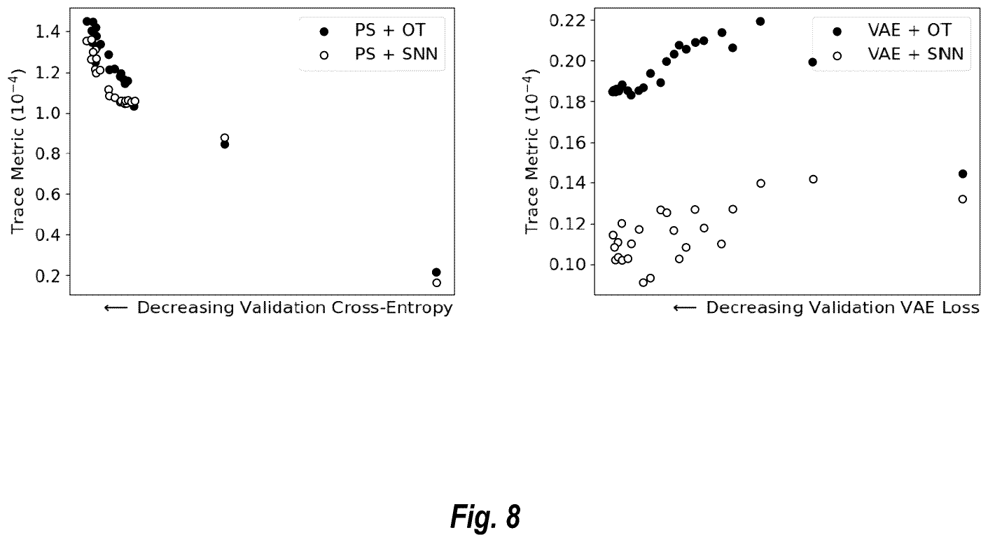

The multi-modal pairing system 100 can utilize a variety of matching approaches. For example, experimenters have analyzed two different matching approaches, shared nearest neighbors (SNN) matching as described in Cao, Z. J. and Gao, G., Multi-omics single-cell data integration and regulatory inference with graph-linked embedding, Nature Biotechnology, 40 (10): 1458-1466, 2022 and optimal transport (OT) matching, see also Villani, C. Optimal transport: old and new, volume 338, Springer, 2009, which is incorporated by reference herein in its entirety. Utilizing the OT matching algorithm with distances defined on the similarity score leads to significant improvement on real world multi-modal matching tasks, which is discussed in more detail below in FIGS. 8-10.

As mentioned above, in some embodiments, the multi-modal pairing system 100 utilizes shared nearest neighbors as the matching algorithm. For instance, using the similarity score, the multi-modal pairing system 100 computes nearest neighbors both within and between the two modalities. In one or more implementations, the multi-modal pairing system 100 computes the normalized shared nearest neighbors (SNN) between each pair of observations as the entry of the matching matrix. For each pair of observations (πᵢ⁽¹⁾, πⱼ⁽²⁾), the multi-modal pairing system 100 can define four sets:
11ᵢⱼ: the k nearest neighbors of πᵢ⁽¹⁾ amongst {πᵢ⁽¹⁾}ᵢ₌₁ⁿ^{₁}. πᵢ⁽¹⁾ is considered a neighbor of itself. In other words, a set within the first modality that identifies the observation as the nearest neighbor of itself.
12ᵢⱼ: the k nearest neighbors of πⱼ⁽²⁾ amongst {πᵢ⁽¹⁾}ᵢ₌₁ⁿ^{₁}. In other words, a set that identifies the nearest neighbor from a second modality to a first modality.
21ᵢⱼ: the k nearest neighbors of πᵢ⁽¹⁾ amongst {πⱼ⁽²⁾}ⱼ₌₁ⁿ^{₂}. In other words, a set that identifies the nearest neighbor from the first modality to the second modality.
22ᵢⱼ: the k nearest neighbors of πⱼ⁽²⁾ amongst {πⱼ⁽²⁾}ⱼ₌₁ⁿ^{₂} is considered a neighbor of itself. In other words, a set within the second modality that identifies the observation as the nearest neighbor of itself.

Intuitively, if πᵢ⁽¹⁾ and πⱼ⁽²⁾ correspond to the same underlying similarity score, their nearest neighbors amongst observations from each modality should be the same. This is measured as a set difference between 11ᵢⱼ and 12ᵢⱼ, and likewise for 21ᵢⱼ and 22ᵢⱼ. Then, a modified Jaccard index is computed as follows, defined as Equation (5):

$${J}_{ij} = \vert {11}_{ij} ⋂ {12}_{ij} \vert + \vert {21}_{ij} ⋂ {22}_{ij} \vert ,$$

Equation (11) indicates the sum of the number of shared neighbors measured in each modality. Then, the multi-modal pairing system 100 can compute the following Jaccard distance to populate the unnormalized matching matrix Equation (6):

$${\tilde{M}}_{ij} = \frac{{J}_{ij}}{4 k - {J}_{ij}} .$$

Note that 4k=|11ᵢⱼ|+|12ᵢⱼ|+|21ᵢⱼ|+|22ᵢⱼ|, since each set contains k distinct neighbors, and thus 0≤\tilde{M}ᵢⱼ≤1, as with the standard Jaccard index. Then, we normalize each row to produce the final matching matrix Equation (7):

$${\tilde{M}}_{ij} = \frac{{\tilde{M}}_{ij}}{{\sum }_{i = 1}^{{n}_{1}} {\tilde{M}}_{ij}}$$

Note that Mᵢⱼ is well defined because πᵢ⁽¹⁾ and πⱼ⁽²⁾ are considered neighbors of themselves.

Lemma A. \tilde{M}ᵢⱼ has at least one non-zero entry in each of its rows and columns for any number of neighbors k≥1.

Proof. Jᵢⱼ>0 for at least one j in each j, which is equivalent to \tilde{M}ᵢⱼ>0. Fix an arbitrary i. 21ᵢⱼ by definition is the same set for every j. By the assumption of k≥1 it is non-empty, so there exists xⱼ⁽²⁾ ∈21ᵢⱼ. Since xⱼ⁽²⁾ is a neighbor of itself, this results in x_{j*}⁽²⁾ ∈ 22_{ij*}, showing that J_{ij*}>0. The same reasoning applied to sets 11 and 12 also shows that Jᵢⱼ for at least one i in each j.

FIG. 5 illustrates optimal transport matching and some assumptions in accordance with one or more embodiments. For instance, a first assumption for OT matching is that t (e.g., a perturbation treatment to a cell) has a non-trivial effect on Z (e.g., the latent space) but does not affect u^{(e)} (e.g., a shared latent value in the shared latent space), which implies that perturbation treatments (e.g., interventions) are able to target a common underlying process without changing modality-specific properties. A second assumption for OT matching is that there is injectivity of the function ƒ^{(e)}.

The first assumption for OT matching is justifiable insofar as modalities represent different measurements of an isolated system, such as in biological studies where the latent space (Z) might correspond to an underlying cell state and modalities refer to different single-cell measurements. In practice, different measurement devices may be more or less sensitive to the biological variation implied by the perturbation treatment (t). The second assumption for OT matching opens the possibility that many different observations can have a shared latent space (z) but can differ by their value in u and the function remains injective (e.g., a rotated image with the exact same content can have a shared z but remain injective due to the rotation being captured in u).

Relaxing Assumption 1 Consider the estimated similarity (e.g., propensity) score of Equation (8):

$$π ( {x}^{( e , t )} ) = P ( t \vert {X}^{( e , t )} = {x}^{( e , t )} )$$

where we do not necessarily require U^{(e)}⊥⊥t|Z^{(t)}, and thus Equation (9):

$$π ( {x}^{( 1 , t )} ) = P ( t \vert {Z}^{( t )} = {z}^{( t )} , {U}^{( 1 )} = {u}^{( 1 )} ) \neq P ( t \vert {Z}^{( t )} = {z}^{( t )} , {U}^{( 2 )} = {u}^{( 2 )} ) = π ( {x}^{( 2 , t )} ) .$$

Suppose that the two observed modalities {πᵢ⁽¹⁾}ᵢ₌₁ⁿ and {πⱼ⁽²⁾}ⱼ₌₁ⁿ are indeed generated by a shared space {zᵢ}ᵢ₌₁ⁿ so that n₁=n₂:=n, but where {πⱼ⁽²⁾}ⱼ₌₁ⁿ is permuted, and with values differing by modality specific information {uᵢ⁽¹⁾}ᵢ₌₁ⁿ^{₁} and {uⱼ⁽²⁾}ⱼ₌₁ⁿ^{₁}. Under the first assumption, we can find πⱼ⁽²⁾=πᵢ⁽¹⁾ for the permuted j.

Matching via optimal transport can allow for relaxation of assumption 1 in a very particular way. First consider the simpler case where t=0,1, so that π can be written in a single dimension, e.g., P(t=1|X^{(e,t)}=x^{(e,t)}. In this case, exact optimal transport matching (e.g., hereinafter referred to as OT matching) on {πᵢ⁽¹⁾}ᵢ₌₁ⁿ and {πⱼ⁽²⁾}ⱼ₌₁ⁿ is equivalent to sorting {πᵢ⁽¹⁾}ᵢ₌₁ⁿ and {πⱼ⁽²⁾}ⱼ₌₁ⁿ, and matching the sorted versions 1-to-1. Under assumption 1, the sorted versions should be exactly equal. A relaxed version of assumption 1 that would still result in the correct ground truth matching is that t affects U⁽¹⁾ and U⁽²⁾ differently, but that the difference is order preserving, or monotone. Denote (πᵢ⁽¹⁾, πᵢ⁽²⁾) as the true pairing, noting that we use the same index i. Consider the following Equation (10):

$$( {π}_{{i}_{1}}^{( 1 )} - {π}_{{i}_{2}}^{( 1 )} ) ( {π}_{{i}_{1}}^{( 2 )} - {π}_{{i}_{2}}^{( 2 )} ) \geq 0 , ∀ {i}_{1} , {i}_{2} = 1 , … , n .$$

The above says that, even if πᵢ⁽¹⁾≠πᵢ⁽²⁾, that their relative orderings will still coincide. Then, exact OT will still recover the ground truth matching. For instance, FIG. 5 provides a visual example of this type of monotonicity. In particular, OT matching allows for t to have different effects on the modality specific information, here uᵢ⁽¹⁾ and uᵢ⁽²⁾, as long as they can be written as transformations that preserve the relative order within modalities. Exact OT matching in 1-d matches according to the relative ordering, and thus exhibits this type of “no crossing” behavior.

For example, suppose that t is a chemical perturbation of a cell, and thus πᵢ⁽¹⁾, πᵢ⁽²⁾ can be seen as a measure of biological response to the perturbation, e.g., in a treated population, xᵢ_{₁}>xᵢ_{₂} indicates samples i₁ had a stronger response than sample i₂, as perceived by the first modality indexed by i. Then, this monotonicity states that the same xᵢ_{₁}>xᵢ_{₂} should be seen in the other modality as well, if the samples i₁ and i₂ truly corresponded to j₁ and j₂.

When t is not a binary treatment and the similarity scores are multidimensional, there is a similar notion, known as cyclic monotonicity. The monotonicity requirement of Equation (10) (e.g., a consistent direction of change as the input increases or decreases) of the function with graph can be denoted as (πᵢ⁽¹⁾, πᵢ⁽²⁾)=∈[0,1]². In higher dimensions, the multi-modal pairing system 100 can ensure the “graph” satisfies the following cyclic monotonicity property:

Definition B. The collection {(πᵢ⁽¹⁾, πᵢ⁽²⁾)}ᵢ₌₁ⁿ is said to be c-cyclically monotone for some cost function c, if for any n=1, . . . , N, and any subset of pairs (πᵢ⁽¹⁾, πᵢ⁽²⁾), . . . , (πᵢ⁽¹⁾, πᵢ⁽²⁾), which gives Equation (11):

$${\sum }_{n = 1}^{N} c ( {π}_{n}^{( 1 )} , {π}_{n}^{( 2 )} ) \leq {\sum }_{n = 1}^{N} c ( {π}_{n}^{( 1 )} , {π}_{n + 1}^{( 2 )} ) .$$

The multi-modal pairing system 100 can define πₙ₊₁=π₁, so that the sequence represents a cycle.

In one or more implementations, the OT cost function is the Euclidean distance, c(x,y)=∥x−y∥₂. The OT matching solution should satisfy cyclic monotonicity. Thus, if the true pairing is uniquely cyclically monotone, the multi-modal pairing system 100 can recover it with OT matching.

Specifically, FIG. 5 illustrates an unobserved sample 500 where the disparate data samples are matched across modalities (e.g., a specific protein expression data matches with a phenomic digital image). For the observed data, FIG. 5 shows a first phenomic digital image 502, a second phenomic digital image 504, a first protein expression data 506, and a second protein expression data 508. Moreover, FIG. 5 also shows a first perturbation classification score 510 for the first phenomic digital image 502, a second perturbation classification score 512 for the second phenomic digital image 504, a third perturbation classification score 514 for the first protein expression data 506, and a fourth perturbation classification score 516 for the second protein expression data 508.

As shown in FIG. 5, the multi-modal pairing system 100 utilizes OT matching by determining the relative value of each of the perturbation classification scores within a modality and matching that across modalities based on the relative value. Specifically, FIG. 5 shows that the multi-modal pairing system 100 determines that the first phenomic digital image 502 matches with the first protein expression data 506 and that the second phenomic digital image 504 matches with the second protein expression data 508.

For instance, the similarity score also allows the multi-modal pairing system 100 to compute a cost function associated with transporting mass between modalities, c(xᵢ⁽¹⁾, xⱼ⁽²⁾)=d′(xᵢ⁽¹⁾, xⱼ⁽²⁾). Let p₁, p₂ denote the uniform distribution over {πᵢ⁽¹⁾}ᵢ₌₁ⁿ^{₁} and {πⱼ⁽²⁾}ⱼ₌₁ⁿ^{₂} respectively. Specifically, the multi-modal pairing system 100 utilizes optimal transport (OT) matching to solve the problem of optimally redistributing mass from p₁ to p₂ in terms of incurring the lowest cost. Thus, OT is a natural way to move between modalities given the similarity score. Let Cᵢⱼ=c(xᵢ⁽¹⁾, xⱼ⁽²⁾) denote the n₁×n₂ cost matrix. The Kantorovich formulation of optimal transport (e.g., an optimal way to move a distribution of mass from one configuration to another, minimizing a given cost) aims to solve the following constrained optimization problem, as defined by Equation (12):

$${min}_{M} {\sum }_{i}^{{n}_{1}} {\sum }_{j}^{{n}_{2}} {C}_{ij} {M}_{ij} , {M}_{ij} \geq 0 ,$$

$$M 1 = {p}_{1} , M 1 = {p}_{2} ,$$

The above notation indicates a linear program, and for n₁=n₂, it can be shown that the optimal solution is a bipartite matching (e.g., a matching optimization problem to find optimal matches between two distinct sets for elements, here the first data modality and the second data modality between {πᵢ⁽¹⁾}ᵢ₌₁ⁿ^{₁} and {πⱼ⁽²⁾}ⱼ₌₁ⁿ^{₂}. The above can be referred to as exact OT. The multi-modal pairing system 100 can add an entropic regularization term (e.g., a measure of uncertainty or randomness as a regularization factor) that ensures smoothness and uniqueness and can be solved efficiently using Sinkhorn's algorithm (e.g., an iterative process to find a coupling that satisfies various constraints and minimizes the entropic regularization term). Entropic OT takes the following form as defined in Equation (13):

$${min}_{M} {\sum }_{i}^{{n}_{1}} {\sum }_{j}^{{n}_{2}} {C}_{ij} {M}_{ij} - Art H ( M ) ,$$

$${M}_{ij} \geq 0 , M 1 = {p}_{1} , M 1 = {p}_{2} ,$$

Where in H(M)=−Σ_{i,j}Mᵢⱼ log (Mᵢⱼ), the entropy of the joint distribution implied by M Entropic OT no longer results in a bipartite matching, which allows for “soft” matches for a point πᵢ⁽¹⁾ corresponding to a set of convex combination weights over {πⱼ⁽²⁾}ⱼ₌₁ⁿ^{₂}. The soft OT approach regularizes towards a higher entropy solution, which has been shown to have statistical benefits, but nonetheless for small enough λ serves as a computationally appealing approximation to exact OT. In other words, the multi-modal pairing system 100 utilizes soft optimal transport matching to relax a strict one-to-one exact match which allows for more flexibility of providing a broader range of possible outcomes for matching unpaired data samples across disparate modalities.

As mentioned above, upon identifying cross-modality pairs, the multi-modal pairing system 100 can utilize these pairs for further machine learning training tasks. FIG. 6 shows the multi-modal pairing system 100 utilizing cross-modality pairs to train a multi-modal machine learning model in accordance with one or more embodiments. As used herein, the term “multi-modal machine learning model learning process” refers to a process of training or learning parameters for the multi-modal machine learning model. Specifically, the multi-modal pairing system 100 generates multi-modal predictions and utilizes the predictions to determine a measure of loss and modify parameters of the multi-modal machine learning model.

As shown, the multi-modal pairing system 100 receives pairs of data samples 600 from the matrix and utilizes a multi-modal machine learning model 602 to process the pairs of data samples 600. As used herein, the term “multi-modal machine learning model” refers to a machine learning model that generates a prediction for a first data modality from a second data modality or vice-versa. For instance, from text data the multi-modal machine learning model can generate a prediction for image data. Specifically, in the context of the multi-modal pairing system 100, once the multi-modal machine learning model 602 is trained, the multi-modal pairing system 100 can generate protein expression data from phenomic digital images or phenomic digital images from protein expression data. In particular, the multi-modal pairing system 100 can train the multi-modal machine learning model 602 capable of generating machine learning representations for different modalities within the same feature space. In some implementations, the multi-modal pairing system 100 can generate embeddings or feature vectors for either data modality into a shared feature space to allow for comparisons between modalities. The multi-modal pairing system 100 can train a variety of different multi-modal machine learning models, such as CLIP or other models such as a multi-modal biological prediction model.

As shown, the multi-modal pairing system 100 generates multi-modal predictions 604 which can include a prediction of a first modality or a second modality. From the multi-modal predictions 604, the multi-modal pairing system 100 can further determine a measure of loss 606 and modify parameters of the multi-modal machine learning model 602 based on the multi-modal predictions 604. As used herein, the term “a measure of loss” refers to a loss function which the multi-modal pairing system 100 attempts to minimize.

In one or more embodiments, the multi-modal pairing system 100 provides a first data sample of a pair of data samples (e.g., one half of the pair) to the multi-modal machine learning model 602 to generate a multi-modal prediction from the data sample. For instance, the multi-modal prediction predicts a second data sample in another data modality from the first data sample (e.g., which is from a first or second modality). In other words, if the multi-modal machine learning model input is from a first data modality, the multi-modal pairing system 100 generates a prediction in a second data modality (or an embedding prediction within a shared feature space that can be compared across data modalities).

Further, in some embodiments, the multi-modal pairing system 100 compares the multi-modal prediction with a second data sample of the pair of data samples (e.g., the second half of the pair that was not provided to the multi-modal machine learning model 602 to generate a prediction or was also used to generate an embedding/feature vector within a shared feature space for comparison). Based on comparing the multi-modal prediction with the second data sample of the pair of data samples, the multi-modal pairing system 100 determines the measure of loss 606. Moreover, the multi-modal machine learning model 602 then modifies parameters of the multi-modal machine learning model 602 (e.g., utilizing back propagation and/or gradient descent) to iteratively improve model predictions across training batches.

In one or more embodiments, the multi-modal pairing system 100 utilizes the matrix discussed in FIG. 5 as part of the multi-modal machine learning model learning process. As mentioned above, the matrix contains entries that indicate probabilities of samples from the second data modality matching samples from the first data modality. For example, the multi-modal pairing system 100 determines a measure of loss using a loss function by using a probability in the matrix as a weight in the loss function. Specifically, the multi-modal pairing system 100 compares a first sample from the first data modality and a second sample from the second data modality utilizing a probability from an entry of the matrix corresponding to the first sample from the first data modality and the second sample from the second data modality as a weight in the loss function. Moreover, the multi-modal pairing system 100 modifies parameters of the multi-modal machine learning model from the multi-modal machine learning model learning process based on the measure of loss determined utilizing the weight.

As mentioned above, the multi-modal pairing system 100 can denote the matrix that defines distribution over datapoints in the form of M_{i,j}=P(xᵢ¹|xⱼ²). The notation for the matrix indicates that the matrix contains entries for data modality i and data modality j. For each entry of the matrix, the matrix contains a probability distribution of a certain data sample of the first data modality matching a certain data sample of the second data modality.

As already mentioned above, the multi-modal pairing system 100 can use the matrix to perform a cross modality prediction for training the multi-modal machine learning model 602. For instance, the multi-modal pairing system 100 can represent a loss function that uses the matrix as:

$$ℒ ( θ ) := {\sum }_{i} {( {x}_{i}^{( 1 )} - {M}_{i} {f}_{θ} ( {x}_{j}^{( 2 )} ) )}^{2} .$$

In the notation for the loss function, f_{θ} represents the multi-modal machine learning model 602 and further indicates that the multi-modal machine learning model 602 is processing a data sample from the second data modality (e.g., the j data modality). From the data sample of the second data modality, the multi-modal machine learning model 602 can generate a prediction for the first data modality. As indicated in the notation for the loss function, the multi-modal pairing system 100 can compare the prediction for the first data modality with the second half of the pair of the data sample from the second data modality weighted by a probability from the matrix Mᵢ. Thus, the multi-modal pairing system 100 utilizes the probability from an entry of the matrix corresponding to the first sample from the first data modality and the second sample from the second data modality as the weight in the loss function to determine the measure of loss 606.

In one or more embodiments, the multi-modal pairing system 100 initiates the multi-modal machine learning model learning process by determining a measure of loss by comparing a multi-modal prediction from the first data modality and two data samples for the second data modality according to corresponding entries in the matrix. In other words, the multi-modal pairing system 100 utilizes a two-sample approach to obtain an unbiased measure of loss for modifying parameters of the multi-modal machine learning model 602.

As used herein, a “two-sample approach” refers to a method of using two samples to get an unbiased estimate. For example, as part of the multi-modal machine learning model learning process, the multi-modal pairing system 100 uses two entries from the matrix based on the plurality of similarity measures. Specifically, the multi-modal pairing system 100 utilizes two entries dependent on two different independent samples to generate the unbiased gradient. Thus, the multi-modal pairing system 100 trains the multi-modal machine learning model 602 from two data samples of the matrix to generate a prediction for a different modality (e.g., generate protein expression data from phenomic digital images or generate phenomic digital images from protein expression data).

To illustrate, the multi-modal pairing system 100 can represent the two-sample approach as

$${∇}_{θ} ℒ ( θ ) \approx - 2 {\sum }_{i} ( {x}_{i}^{( 1 )} - {f}_{θ} ( {\dot{x}}_{j}^{( 2 )} ) ) {∇}_{θ} {f}_{θ} ( {\ddot{x}}_{j}^{( 2 )} ) \cdot {\dot{x}}_{j}^{( 2 )} , {\ddot{x}}_{j}^{( 2 )} ∼ {M}_{i , j} .$$

In the two-sample approach notation, the multi-modal pairing system 100 determines an unbiased gradient or a measure of loss. For instance, the notation shows f_{θ} (e.g., the multi-modal machine learning model 602) processing a first data sample from the second data modality (\dot{x}ⱼ⁽²⁾) and f_{θ} further processing a second data sample from the second data modality (\ddot{x}ⱼ⁽²⁾). Specifically, the multi-modal pairing system 100 can utilize the two-sample approach to generate a data sample prediction in the first data modality (e.g., a difference between a prediction and an actual) and compare the difference with the second data sample from the second data modality (e.g., the Jacobian term which typically represents a rate of change with multiple variables within a matrix).

As mentioned, the multi-modal pairing system 100 trains the multi-modal machine learning and at inference time, the multi-modal machine learning model can predict one modality from another modality. FIG. 7 illustrates that the multi-modal pairing system 100 can receive phenomic digital image 700 and utilize a multi-modal machine learning model 702 to generate protein expression data 704 from the phenomic digital image 700. Moreover, FIG. 7 also illustrates that the multi-modal pairing system 100 can receive protein expression data 704 and utilize the multi-modal machine learning model 702 to generate a phenomic digital image 706.

The multi-modal pairing system 100 can train a variety of multi-modal models. Specifically, the multi-modal pairing system 100 can train a multi-modal model in a feature space or the multi-modal pairing system 100 can train a multi-modal using end-to-end learning (e.g., without explicitly derived features). Accordingly, the multi-modal pairing system 100 can predict directly from a first modality to a second modality, from a second modality to a first modality, or generate an embedding from a first modality and/or a second modality into a shared feature space (e.g., so that similarity or differences between multiple modalities can be observed and measured within the shared feature space).

The following description provides some details for experimental testing of one or more experimental embodiments of the multi-modal pairing system 100 training a multi-modal machine learning model. For example, experimenters have evaluated experimental implementations of the multi-modal pairing system 100 on various datasets. For instance, experimenters have tested the multi-modal pairing system 100 on a synthetic interventional image dataset. Further, experimenters have also tested the multi-modal pairing system 100 on a real-world single-cell CITE-seq data (e.g., cellular indexing of transcriptomes and epitopes by sequencing that combines single-cell RNA sequencing with protein expression profiling), which allows for a small number of cell surface proteins to be measured simultaneously to RNA sequencing. Note the CTIE-seq dataset is not interventional-experimenters use the cell type as the classification target t instead. In both cases the ground truth matching is known to make evaluation possible but hidden during training.

For the CITE-seq data, the experimenters utilize a graph-linked VAE (e.g., variational autoencoder) (scGLUE-single-cell graph learning using embedding), which has access to specific biological metadata that connects the biological modalities, for matching. For an additional approach, experimenters also utilize the VAE approach developed in Yang, K. D., Belyaeva, A., Venkatachalapathy, S., Damodaran, K., Katcoff, A., Radhakrishnan, A., Shivashankar, G., and Uhler, C. Multi-domain translation between single-cell imaging and sequencing data using autoencoders, Nature communications, 12 (1): 31, 2021 (hereinafter “Yang”), which also uses the grouping variable as additional information by learning a classifier from the latent space. The resulting latent space hence carries similar information to the similarity score. However, the goal there is to translate between different modalities, therefore requiring an encoder-decoder structure. Under one or more assumptions discussed above, the approach that modalities be reconstructed introduces information from the modality specific factors U(1) and U(2), which the multi-modal pairing system 100 can ignore by computing the similarity score to target the shared latent space Z. For both scGLUE and the VAE, experimenters used both SNN and OT to perform the final matching in the latent space.

For both datasets, experimenters used the general architecture of a linear classification head on top of a suitable encoder. The details of this encoder can be the same between the VAE and similarity score methods. For other methods, experimenters used existing implementations with suggested default settings. For the encoder, in the image dataset, experimenters used the convolutional neural network used in Yang, and for the CITE-seq dataset, experimenters used fully-connected multi-layer perceptron's for both RNA (top 200 principal component analysis) and protein data as encoders. Optimization was performed using Adam with a one-cycle learning rate scheduler. Experimenters used a measure of loss (e.g., cross-entropy loss) to train classifiers for similarity score estimation.

Both SNN and OT use the Euclidean distance function to determine neighbors and compute the cost matrix, respectively. SCOT (single-cell optimal transport) uses the correlation distance by default, and experimenters found that this resulted in better performance than Euclidean distance. Experimenters used a single neighbor for SNN matching, which resulted in the best performance. Both SCOT and OT solve the entropic regularized OT, and for these experimenters used a regularization parameter of 0.05.

Experimenters can evaluate how well samples are matched using the ground truth provided by the datasets. In these cases, the dataset sizes are necessarily balanced, so that n=n₁=n₂. In each case, the metric is a function of a n×n matching matrix M.

Further, experimenters use a trace metric which refers to a measure or distance defined using a trace of a matrix. For instance, a trace indicates a sum of the diagonal elements of a square matrix. The trace metric can help experimenters assess the similarity or difference between matrices. Assuming the given indices correspond to the true matching (i.e. xᵢ⁽¹⁾, corresponds to xᵢ⁽²⁾), the multi-modal pairing system 100 can compute the average weight on correct matches, which is the normalized trace of M:

$$\frac{1}{n} Tr ( M ) = \frac{1}{n} {\sum }_{i = 1}^{n} {M}_{ii} .$$

As a baseline, notice that a uniformly random matching that assigns Mᵢⱼ=1/n for each cell yields Tr(M)=1 and hence will obtain a metric of 1/n. This metric however does not capture potential failure modes of matching. For example, exactly matching one sample, while adversarially matching dissimilar-samples for the remainder also yields a trace of 1/n, which is equal to that of a random matching.

Because the image dataset is synthetic, experimenters could access the ground truth latent values that generated the images, z={zᵢ}ᵢ₌₁ⁿ. Experimenters computed matched latents as Mz, the projection according to the matching matrix. Then, to evaluate the quality of the matching, experimenters computed the mean square error, hereinafter MSE:

$$MSE ( M ) = \frac{1}{n} {‖ z - M z ‖}_{2}^{2} .$$

For the CITE-seq dataset, experimenters used the Fraction Of Samples Closer Than the True Match (FOSCTTM) (as described in Demetci et al., 2022; Liu et al., 2019) as an alternative matching metric. First, experimenters distributed the mass of x⁽²⁾={πᵢ⁽¹⁾}ᵢ₌₁ⁿ^{₁} to x⁽¹⁾={πⱼ⁽²⁾}ⱼ₌₁ⁿ^{₂} as \hat{x}⁽¹⁾=Mx⁽²⁾, resulting in a projection of the first modality x⁽¹⁾ in the space of the second modality. Then, experimenters computed a cross-modality distance as follows. For each point in \hat{x}⁽¹⁾, experimenters computed the Euclidean distance to each point in x⁽¹⁾, and computed the fraction of samples in x⁽¹⁾ that are closer than the true match. Experimenters also repeated this for each point in x⁽¹⁾, computing the fraction of samples in \hat{x}⁽¹⁾ in this case. That is, assuming again that the given indices correspond to the true matching, the experimenters compute:

$$FOSCTTM ( M ) = \frac{1}{2 n} [ {\sum }_{i = 1}^{n} ( \frac{1}{n} {\sum }_{j \neq i} \{ d ( {\hat{x}}^{( 1 )} , {x}^{( 1 )} ) < d ( {\hat{x}}^{( 1 )} , {x}^{( 1 )} ) \} ) + {\sum }_{j = 1}^{n} ( \frac{1}{n} {\sum }_{i \neq j} 1 \{ d ( {x}^{( 1 )} , {\hat{x}}^{( 1 )} ) < d ( {x}^{( 1 )} , {\hat{x}}^{( 1 )} ) \} ) ,$$

where the above notations are functions of M through the computation \hat{x}⁽¹⁾=M x⁽²⁾. As a baseline, completely random matching would be expected, when distances between points are randomly distributed, to have an FOSCTTM of 0.5.

Finally, experimenters considered one metric that examines whether matched samples are useful for downstream tasks. For this, experimenters chose the cross-modality prediction task of predicting protein levels from RNA expression in the CITE-seq dataset. To do this, experimenters trained the same supervised learning model (e.g., a 2-layer multi-layer perceptron with MSE loss) on three different datasets. First, experimenters trained on the ground truth matching to determine the optimal performance expected from an artificially matched dataset. Then, experimenters trained on a dataset corresponding to a matching matrix M, which was obtained by sampling, for each xᵢ⁽¹⁾, an xᵢ⁽²⁾ proportional to the row Mᵢ. Finally, as a baseline, experimenters trained on a dataset produced by a uniform sampling of xᵢ⁽²⁾.

FIG. 8 illustrates experimental results of how validation cross-entropy has an inverse relationship with ground truth matching while the same pattern is not present with the variational autoencoder. For example, the improvements of the multi-modal pairing system 100 include its ability to learn the conditional distributions p(t|Xᵢ^{(e)}). For instance, as shown in FIG. 8, the validation cross entropy is a computable metric that can thus serve as a monitor for the matching performance. Specifically, the indication of the validation cross-entropy having an inverse relationship with ground truth matching is validated in conducted experiments, even on real CITE-seq data, where experimenters saw that a lower validation loss typically corresponds to higher matching performance (e.g., graph on the left), which is not the case for the VAE (e.g., graph on the right).

Experimenters performed the matching step within observations with the same t, producing a matching matrix, and hence matching metrics, within each group (with the exception of modality prediction). Experimenters reported average, minimum, and maximum (over t). For the experimental embodiment of the multi-modal pairing system 100 and the VAE, researchers checkpointed the model at the lowest validation loss and reported their metrics. Note this is not necessarily the model that exhibits optimal matching but is the best model (in this case) to select without validating against ground truth, as shown in FIG. 8.

In particular, FIG. 8 illustrates VAE and classifier validation metrics on the CITE-seq dataset. Notice that validation cross-entropy inversely tracks the ground truth matching metrics, and thus can be used as a proxy in practical settings where the ground truth is unknown. The same pattern does not hold for the VAE, see Yang.

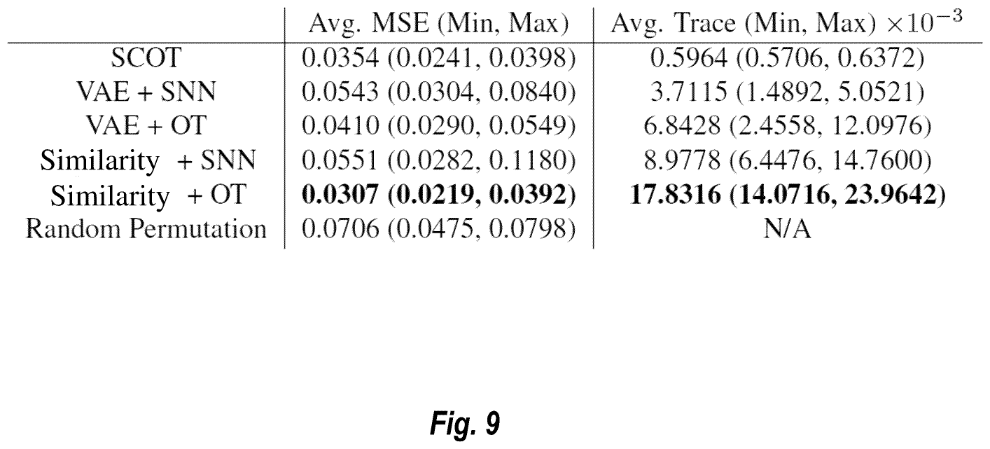

FIG. 9 illustrates results on synthetic data. There are 12 groups corresponding to interventions on the latent position, with approximately 1700 observations per group. A trace of 10 for example corresponds to a total weight of 10° ø 10-3°ø 1700=17 on the true matching, out of 1700. As indicated by the bolded numbers in FIG. 9, the average MSE indicates that the similarity score with OT has the best average MSE. Likewise for the average trace, the similarity score with OT has the best average trace. Thus, FIG. 9 illustrates the benefits of using the OT matching to match unpaired data samples.

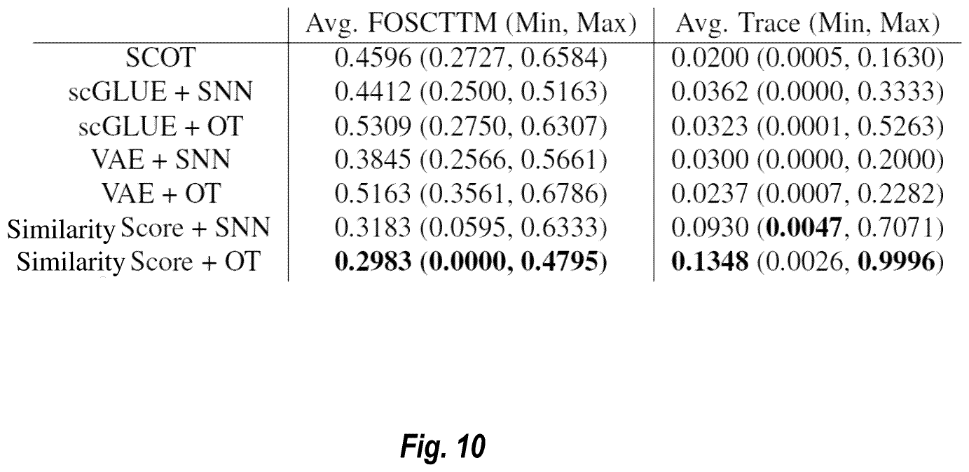

FIG. 10 illustrates results on CITE-seq data. There are a total of 45 cell types (groups) with a varying number of observations per group. As such, the average trace is difficult to interpret (for example, the maximum trace of 0.9996 corresponds to a near-perfect matching within a group with only 7 cells), but notably OT matching on similarity scores outperforms the other methods within each group as well as on average.

scGLUE is trained according to the public implementation, which uses learning rate reduction and early stopping strategies. SCOT is a non-iterative approach and thus experimenters directly reported its results. OT matching on similarity scores consistently outperforms other methods, typically followed by SNN matching on similarity scores. Curiously, SCOT performs well on the MSE metric for image data, but only places slightly above random in the trace metric. This indicates that it matches scenes with similar latent coordinates, without placing any significant weight on the exact ground truth. This suggests that exact matches may not be necessary for a method to be useful for downstream tasks.

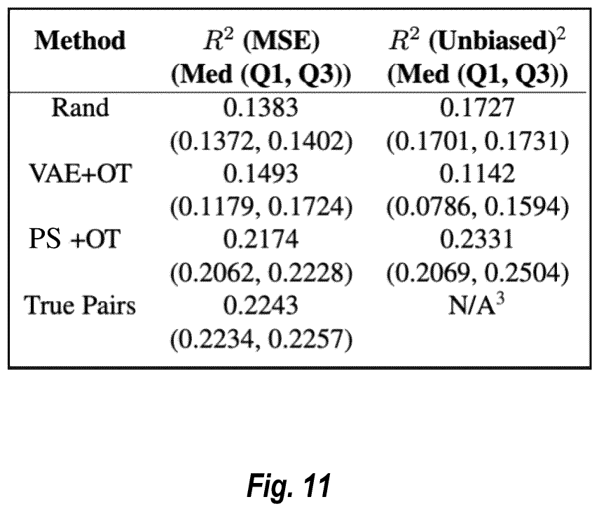

FIG. 11 illustrates the benefits of the multi-modal pairing system 100 utilizing a two-sample approach (e.g., to determine an unbiased gradient or measure of loss). For example, for the similarity score plus OT for MSE, an R² score (e.g., a statistical measure that represents the proportion of the variance in a dependent variable that is predictable from independent variables, in other words, how well a model's predictions match the observed data) of 0.2174 while the R² score for the two-sample approach (e.g., unbiased approach) shows a score of 0.2331. Accordingly, the multi-modal pairing system 100 utilizing the two-sample approach results in marginal improvements (E.g., an R² increase of around 2%) for matching unpaired data samples.

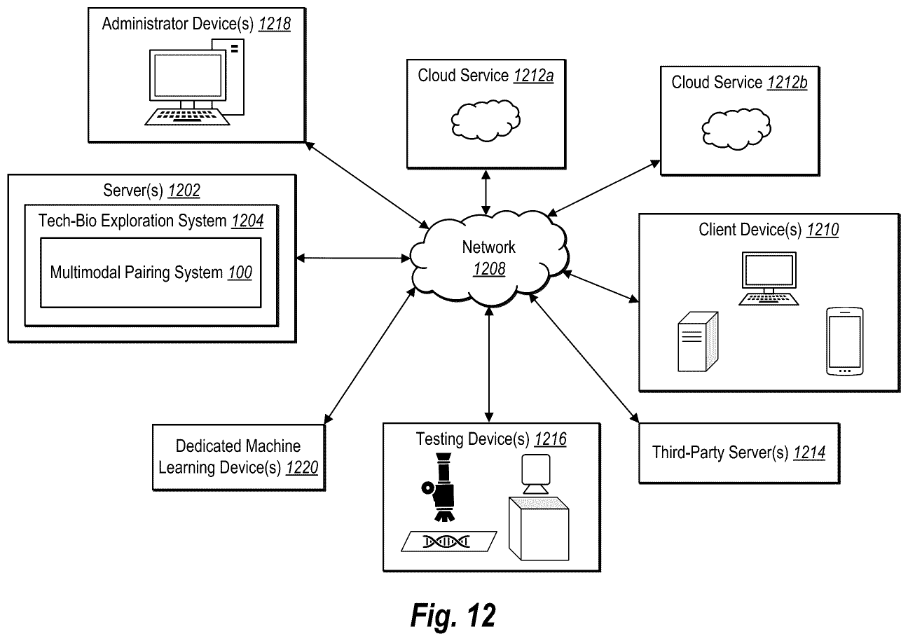

Additional detail regarding a multi-modal pairing system 100 will now be provided with reference to the figures. In particular, FIG. 12 illustrates a schematic diagram of a system environment in which the multi-modal pairing system 100 can operate in accordance with one or more embodiments.

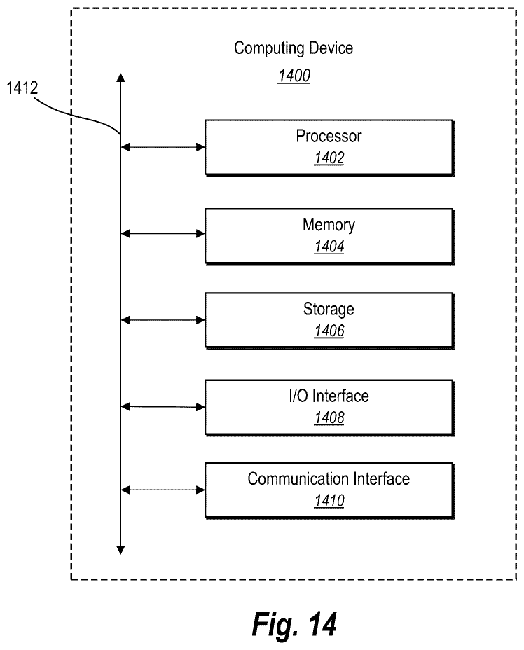

As shown in FIG. 12, the environment includes server(s) 1202 (which includes a tech-bio exploration system 1204 and the multi-modal pairing system 100), a network 1208, client device(s) 1210, cloud service(s) 1212a-1212b, third-party server(s) 1214, testing device(s) 1216, administrator device(s) 1218, and dedicated machine learning device(s) 1220. As further illustrated in FIG. 12, the various computing devices within the environment can communicate via the network 1208. Although FIG. 12 illustrates the multi-modal pairing system 100 being implemented by a particular component and/or device within the environment, the multi-modal pairing system 100 can be implemented, in whole or in part, by other computing devices and/or components in the environment (e.g., the administrator device(s) 1218, the client device(s) 1210). Additional description regarding the illustrated computing devices is provided with respect to FIG. 14 below.

As shown in FIG. 12, the server(s) 1202 (e.g., one or more local servers operated by a particular entity) can include the tech-bio exploration system 1204. In some embodiments, the tech-bio exploration system 1204 can determine, store, generate, and/or display tech-bio information including maps of biology, biology experiments from various sources, and/or machine learning tech-bio predictions. For instance, the tech-bio exploration system 1204 can analyze data signals corresponding to various treatments or interventions (e.g., compounds or biologics) and the corresponding relationships in genetics, protenomics, phenomics (i.e., cellular phenotypes), and invivomics (e.g., expressions or results within a living animal).

For instance, the tech-bio exploration system 1204 can generate and access experimental results corresponding to gene sequences, protein shapes/folding, protein/compound interactions, phenotypes resulting from various interventions or perturbations (e.g., gene knockout sequences or compound treatments), and/or invivo experimentation on various treatments in living animals. By analyzing these signals (e.g., utilizing various machine learning models), the tech-bio exploration system 1204 can generate or determine a variety of predictions and inter-relationships for improving treatments/interventions.

To illustrate, the tech-bio exploration system 1204 can generate maps of biology indicating biological inter-relationships or similarities between these various input signals to discover potential new treatments. For example, the tech-bio exploration system 1204 can utilize machine learning and/or maps of biology to identify a similarity between a first gene associated with disease treatment and a second gene previously unassociated with the disease based on a similarity in resulting phenotypes from gene knockout experiments. The tech-bio exploration system 1204 can then identify new treatments based on the gene similarity (e.g., by targeting compounds the impact the second gene). Similarly, the tech-bio exploration system 1204 can analyze signals from a variety of sources (e.g., protein interactions, or invivo experiments) to predict efficacious treatments based on various levels of biological data.

The tech-bio exploration system 1204 can generate GUIs comprising dynamic user interface elements to convey tech-bio information and receive user input for intelligently exploring tech-bio information. Indeed, as mentioned above, the tech-bio exploration system 1204 can generate GUIs displaying different maps of biology that intuitively and efficiently express complex interactions between different biological systems for identifying improved treatment solutions. Furthermore, the tech-bio exploration system 1204 can also electronically communicate tech-bio information between various computing devices.

As shown in FIG. 12, the tech-bio exploration system 1204 can include a system that facilitates various models or algorithms for generating maps of biology (e.g., maps or visualizations illustrating similarities or relationships between genes, proteins, diseases, compounds, and/or treatments) and discovering new treatment options over one or more networks. For example, the tech-bio exploration system 1204 collects, manages, and transmits data across a variety of different entities, accounts, and devices. In some cases, the tech-bio exploration system 1204 is a network system that facilitates access to (and analysis of) tech-bio information within a centralized operating system. Indeed, the tech-bio exploration system 1204 can link data from different network-based research institutions to generate and analyze maps of biology.

As shown in FIG. 12, the tech-bio exploration system 1204 can include a system that comprises the multi-modal pairing system 100 that generates, stores, manages, transmits, and analyzes machine learning model datasets. For example, the multi-modal pairing system 100 can generate multi-modal machine learning training pairs. In particular, the multi-modal pairing system 100 can utilize trained machine learning models (e.g., classification models) to generate sets of perturbation classification scores and compare the perturbation classification scores to determine similarity measures. Further, the multi-modal pairing system utilizes the similarity measures to match unpaired multi-modal data to generate multi-modal machine learning training pairs. The multi-modal pairing system 100 can then utilizes the multi-modal machine learning pairs to train a multi-modal machine learning model (e.g., to generate multi-modal embeddings from disparate data sources, such as phenomics data, protenomics data (e.g., protein expression data or RNA sequencing data), and/or invivomics data).

As used herein, the term “machine learning model” includes a computer algorithm or a collection of computer algorithms that can be trained and/or tuned based on inputs to approximate unknown functions. For example, a machine learning model can include a computer algorithm with branches, weights, or parameters that changed based on training data to improve for a particular task. Thus, a machine learning model can utilize one or more learning techniques (e.g., supervised or unsupervised learning) to improve in accuracy and/or effectiveness. Example machine learning models include various types of decision trees, support vector machines, Bayesian networks, random forest models, or neural networks (e.g., deep neural networks, generative adversarial neural networks, convolutional neural networks, recurrent neural networks, or diffusion neural networks). Similarly, the term “machine learning data” refers to information, data, or files generated or utilized by a machine learning model. Machine learning data can include training data, machine learning parameters, or embeddings/predictions generated by a machine learning model.

As also illustrated in FIG. 12, the environment includes the client device(s) 1210. For example, the client device(s) 1210 may include, but is not limited to, a mobile device (e.g., smartphone, tablet) or other type of computing device, including those explained below with reference to FIG. 7. Additionally, the client device(s) 1210 can include a computing device associated with (and/or operated by) user accounts for the tech-bio exploration system 1204. Moreover, the environment can include various numbers of client devices that communicate and/or interact with the tech-bio exploration system 1204 and/or the multi-modal pairing system 100.

Furthermore, in one or more implementations, the client device(s) 1210 includes a client application. The client application can include instructions that (upon execution) cause the client device(s) 1210 to perform various actions. For example, a user of a user account can interact with the client application on the client device(s) 1210 to access tech-bio information, initiate a request for a machine learning dataset, initiate training of a machine learning model utilizing a machine learning dataset, and/or generate GUIs comprising a machine learning dataset, machine learning predictions/results, and/or machine learning efficacy.

As further shown in FIG. 12, the environment includes the network 1208. As mentioned above, the network 1208 can enable communication between components of the environment. In one or more embodiments, the network 1208 may include a suitable network and may communicate using a various number of communication platforms and technologies suitable for transmitting data and/or communication signals, examples of which are described with reference to FIG. 14. Furthermore, although FIG. 12 illustrates computing devices communicating via the network 1208, the various components of the environment can communicate and/or interact via other methods (e.g., communicate directly).

As mentioned previously, in one or more implementations, the multi-modal pairing system 100 generates and accesses machine learning objects, such as results from biological assays. As shown, in FIG. 12, the multi-modal pairing system 100 can communicate with testing device(s) 1216 to obtain and then store this information. For example, the tech-bio exploration system 1204 can interact with the testing device(s) 1216 that include intelligent robotic devices and camera devices for generating and capturing digital images of cellular phenotypes resulting from different perturbations (e.g., genetic knockouts or compound treatments of stem cells) and sequencing machines. Similarly, the testing device(s) can include camera devices and/or other sensors (e.g., heat or motion sensors) capturing real-time information from animals as part of invivo experimentation. The tech-bio exploration system 1204 can also interact with a variety of other testing device(s) such as devices for determining, generating, or extracting gene sequences or protein information.

As shown in FIG. 12, the environment also includes a variety of computing devices (i.e., digital repository platforms) capable of storing machine learning data objects. For instance, the multi-modal pairing system 100 can store sets of perturbation classification scores on digital repository platforms for later analysis to determine multi-modal machine learning training pairs. As used herein, the term digital repository platform includes a storage device or set of storage devices (e.g., for storing digital files corresponding to machine learning datasets). In particular, a digital repository platform can include a set of storage devices at a particular location or controlled by a particular entity. Thus, for example, a digital repository platform can include a cloud service (e.g., Amazon Web Services), a local server, or a third-party server.

For example, with regard to the server(s) 1202, local servers operating the tech-bio exploration system 1204 can store machine learning data objects on various servers distributed geographically across different parts of the country or world. In addition, the cloud service(s) 1212a-1212b can also store machine learning data objects. For example, the multi-modal pairing system 100 can utilize a cloud storage service provider and transmit machine learning data objects to the cloud service(s) 1212a-1212b. Further, the multi-modal pairing system 100 can interact with third-party server(s) 1214 (e.g., servers operated and owned by separate entities, such as a coordinating partner with its own biological data). The multi-modal pairing system 100 can collaborate with third parties to generate machine learning datasets from machine learning data objects retained on the third-party server(s) 1214. In addition, the multi-modal pairing system 100 can also interact with dedicated machine learning device(s) 1220. For example, the dedicated machine learning device(s) 1220 can include computing devices or virtual machines dedicated to training or implementing large-scale machine learning models. In some implementations, the multi-modal pairing system 100 can also store machine learning data objects on the dedicated machine learning device(s) 1220. For instance, the dedicated machine learning device(s) 1220 can include a first classification model for generating perturbation classification scores using a phenomic image classification model. Further, the dedicated machine learning device(s) 1220 can also include a second classification model for generating perturbation classification scores using a protein expression classification model.

As shown in FIG. 12, the environment also includes administrator device(s) 1218. For example, the multi-modal pairing system 100 can utilize the administrator device(s) 1218 to control various functions or operations in scheduling or implementing assays, training or implementing machine learning models, receiving and responding to requests, and/or managing a compound/drug discovery pipeline. To illustrate, the administrator device(s) 1218 can identify assays, set up machine learning processes, determine a framework or pipeline for analyzing machine learning models, selecting storage locations in particular digital repository platforms for digital files, and/or determine access permissions to particular digital information.

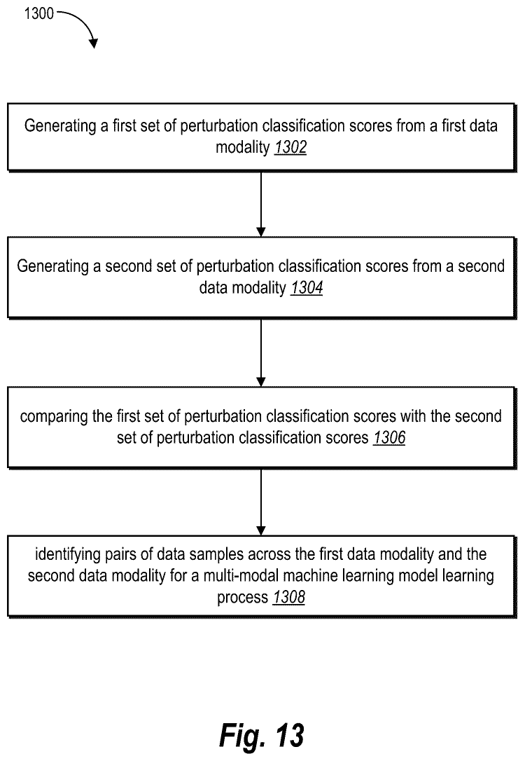

FIGS. 1-12, the corresponding text, and the examples provide a number of different systems, methods, and non-transitory computer readable media for generating perturbation classification scores, comparing the perturbation classification scores, and identifying pairs of data samples across disparate modalities. In addition to the foregoing, embodiments can also be described in terms of flowcharts comprising acts for accomplishing a particular result. For example, FIG. 13 illustrates a flowchart of an example sequence of acts in accordance with one or more embodiments.

While FIG. 12 illustrates acts according to some embodiments, alternative embodiments may omit, add to, reorder, and/or modify any of the acts shown in FIG. 13. The acts of FIG. 13 can be performed as part of a method (e.g., a computer-implemented method). Alternatively, a non-transitory computer readable medium can comprise instructions, that when executed by one or more processors (e.g., at least one processor), cause a computing device to perform the acts of FIG. 13. In still further embodiments, a system can perform the acts of FIG. 13. Additionally, the acts described herein may be repeated or performed in parallel with one another or in parallel with different instances of the same or other similar acts.

FIG. 13 illustrates an example series of acts 1300 for identifying pairs of data samples across disparate data modalities for a multi-modal machine learning model learning process in accordance with one or more embodiments. The series of acts 1300 can include an act 1302 of generating a first set of perturbation classification scores from a first data modality, an act 1304 of generating a second set of perturbation classification scores from a second data modality, an act 1306 of comparing the first set of perturbation classification scores with the second set of perturbation classification scores, and an act 1308 of identifying pairs of data samples across the first data modality and the second data modality for a multi-modal machine learning model learning process. Specifically, the series of acts 1300 can include acts 1302-1308 of generating, utilizing a first classification model, a first set of perturbation classification scores from a first data modality; generating, utilizing a second classification model, a second set of perturbation classification scores from a second data modality; comparing the first set of perturbation classification scores with the second set of perturbation classification scores to determine a plurality of similarity measures; and identifying, based on the plurality of similarity measures, pairs of data samples across the first data modality and the second data modality for a multi-modal machine learning model learning process utilizing the pairs of data samples.

For example, in one or more embodiments, the series of acts 1300 includes phenomic digital images and generating the first set of perturbation classification scores from the first data modality comprises generating, utilizing a phenomic image classification model, the first set of perturbation classification scores from a phenomic digital image of a cell exposed to a perturbation treatment. In one or more implementations, the series of acts 1300 includes protein expression data and generating the second set of perturbation classification scores from the second data modality comprises generating, utilizing a protein expression classification model, the second set of perturbation classification scores from a protein expression measurement of a cell exposed to a perturbation treatment.

In addition, in one or more implementations, the series of acts 1300 includes determining the plurality of similarity measures comprises determining a cross-modality distance within a feature space between a first perturbation of the first data modality and a second perturbation of the second data modality; and identifying, utilizing a matching algorithm, the pairs of data samples across the first data modality and the second data modality based on the cross-modality distance.

Further, in some implementations, the series of acts 1300 includes generating, utilizing a multi-modal machine learning model from the multi-modal machine learning model learning process, multi-modal predictions for the pairs of data samples; and modifying parameters of the multi-modal machine learning model utilizing a measure of loss determined based on the pairs of data samples.

In one or more implementations, the series of acts 1300 includes utilizing the multi-modal machine learning model to generate protein expression data from phenomic digital images or generate phenomic digital images from protein expression data. Moreover, in one or more implementations, the series of acts 1300 includes initiating the multi-modal machine learning model learning process utilizing the pairs of data samples by generating a matrix comprising entries that indicate probabilities of samples from the second data modality matching samples from the first data modality.

In addition, in some implementations, the series of acts 1300 includes utilizing the matrix to initiate the multi-modal machine learning model learning process by determining, utilizing a loss function, a measure of loss by comparing a first sample from the first data modality and a second sample from the second data modality utilizing a probability from an entry of the matrix corresponding to the first sample from the first data modality and the second sample from the second data modality as a weight in the loss function; and modifying parameters of a multi-modal machine learning model from the multi-modal machine learning model learning process based on the measure of loss determined utilizing the weight.

In one or more implementations, the series of acts 1300 includes utilizing the matrix to initiate the multi-modal machine learning model learning process by determining a measure of loss by comparing a multi-modal prediction from the first data modality and two data samples for the second data modality according to corresponding entries in the matrix.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., memory), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed by a processor, cause a general-purpose computer, special purpose computer, or special purpose processing device to perform a certain function or group of functions. In some embodiments, computer-executable instructions are executed by a general-purpose computer to turn the general-purpose computer into a special purpose computer implementing elements of the disclosure. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. As used herein, the term “cloud computing” refers to a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In addition, as used herein, the term “cloud-computing environment” refers to an environment in which cloud computing is employed.

FIG. 14 illustrates a block diagram of an example computing device 1400 that may be configured to perform one or more of the processes described above. One will appreciate that one or more computing devices, such as the computing device 1400 may represent the computing devices described above. In one or more embodiments, the computing device 1400 may be a mobile device (e.g., a mobile telephone, a smartphone, a PDA, a tablet, a laptop, a camera, a tracker, a watch, a wearable device, etc.). In some embodiments, the computing device 1400 may be a non-mobile device (e.g., a desktop computer or another type of client device). Further, the computing device 1400 may be a server device that includes cloud-based processing and storage capabilities.

As shown in FIG. 14, the computing device 1400 can include one or more processor(s) 1402, memory 1404, a storage device 1406, input/output interfaces 1408 (or “I/O interfaces 1408”), and a communication interface 1410, which may be communicatively coupled by way of a communication infrastructure (e.g., bus 1412). While the computing device 1400 is shown in FIG. 14, the components illustrated in FIG. 14 are not intended to be limiting. Additional or alternative components may be used in other embodiments. Furthermore, in certain embodiments, the computing device 1400 includes fewer components than those shown in FIG. 14. Components of the computing device 1400 shown in FIG. 14 will now be described in additional detail.

In particular embodiments, the processor(s) 1402 includes hardware for executing instructions, such as those making up a computer program. As an example, and not by way of limitation, to execute instructions, the processor(s) 1402 may retrieve (or fetch) the instructions from an internal register, an internal cache, memory 1404, or a storage device 1406 and decode and execute them.

The computing device 1400 includes memory 1404, which is coupled to the processor(s) 1402. The memory 1404 may be used for storing data, metadata, and programs for execution by the processor(s). The memory 1404 may include one or more of volatile and non-volatile memories, such as Random-Access Memory (“RAM”), Read-Only Memory (“ROM”), a solid-state disk (“SSD”), Flash, Phase Change Memory (“PCM”), or other types of data storage. The memory 1404 may be internal or distributed memory.

The computing device 1400 includes a storage device 1406 includes storage for storing data or instructions. As an example, and not by way of limitation, the storage device 1406 can include a non-transitory storage medium described above. The storage device 1406 may include a hard disk drive (HDD), flash memory, a Universal Serial Bus (USB) drive or a combination these or other storage devices.

As shown, the computing device 1400 includes one or more I/O interfaces 1408, which are provided to allow a user to provide input to (such as user strokes), receive output from, and otherwise transfer data to and from the computing device 1400. These I/O interfaces 1408 may include a mouse, keypad or a keyboard, a touch screen, camera, optical scanner, network interface, modem, other known I/O devices or a combination of such I/O interfaces 1408. The touch screen may be activated with a stylus or a finger.

The I/O interfaces 1408 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O interfaces 1408 are configured to provide graphical data to a display for presentation to a user. The graphical data may be representative of one or more graphical user interfaces and/or any other graphical content as may serve a particular implementation.

The computing device 1400 can further include a communication interface 1410. The communication interface 1410 can include hardware, software, or both. The communication interface 1410 provides one or more interfaces for communication (such as, for example, packet-based communication) between the computing device and one or more other computing devices or one or more networks. As an example, and not by way of limitation, communication interface 1410 may include a network interface controller (NIC) or network adapter for communicating with an Ethernet or other wire-based network or a wireless NIC (WNIC) or wireless adapter for communicating with a wireless network, such as a WI-FI. The computing device 1400 can further include a bus 1412. The bus 1412 can include hardware, software, or both that connects components of computing device 1400 to each other.

In one or more implementations, various computing devices can communicate over a computer network. This disclosure contemplates any suitable network. As an example, and not by way of limitation, one or more portions of a network may include an ad hoc network, an intranet, an extranet, a virtual private network (“VPN”), a local area network (“LAN”), a wireless LAN (“WLAN”), a wide area network (“WAN”), a wireless WAN (“WWAN”), a metropolitan area network (“MAN”), a portion of the Internet, a portion of the Public Switched Telephone Network (“PSTN”), a cellular telephone network, or a combination of two or more of these.

In particular embodiments, the computing device 1400 can include a client device that includes a requester application or a web browser, such as MICROSOFT INTERNET EXPLORER, GOOGLE CHROME, or MOZILLA FIREFOX, and may have one or more add-ons, plug-ins, or other extensions, such as TOOLBAR or YAHOO TOOLBAR. A user at the client device may enter a Uniform Resource Locator (“URL”) or other address directing the web browser to a particular server (such as server), and the web browser may generate a Hyper Text Transfer Protocol (“HTTP”) request and communicate the HTTP request to server. The server may accept the HTTP request and communicate to the client device one or more Hyper Text Markup Language (“HTML”) files responsive to the HTTP request. The client device may render a webpage based on the HTML files from the server for presentation to the user. This disclosure contemplates any suitable webpage files. As an example, and not by way of limitation, webpages may render from HTML files, Extensible Hyper Text Markup Language (“XHTML”) files, or Extensible Markup Language (“XML”) files, according to particular needs. Such pages may also execute scripts such as, for example and without limitation, those written in JAVASCRIPT, JAVA, MICROSOFT SILVERLIGHT, combinations of markup language and scripts such as AJAX (Asynchronous JAVASCRIPT and XML), and the like. Herein, reference to a webpage encompasses one or more corresponding webpage files (which a browser may use to render the webpage) and vice versa, where appropriate.

In particular embodiments, the tech-bio exploration system 1204 may include a variety of servers, sub-systems, programs, modules, logs, and data stores. In particular embodiments, the tech-bio exploration system 1204 may include one or more of the following: a web server, action logger, API-request server, transaction engine, cross-institution network interface manager, notification controller, action log, third-party-content-object-exposure log, inference module, authorization/privacy server, search module, user-interface module, user-profile (e.g., provider profile or requester profile) store, connection store, third-party content store, or location store. The tech-bio exploration system 1204 may also include suitable components such as network interfaces, security mechanisms, load balancers, failover servers, management-and-network-operations consoles, other suitable components, or any suitable combination thereof. In particular embodiments, the tech-bio exploration system 1204 may include one or more user-profile stores for storing user profiles and/or account information for credit accounts, secured accounts, secondary accounts, and other affiliated financial networking system accounts. A user profile may include, for example, biographic information, demographic information, financial information, behavioral information, social information, or other types of descriptive information, such as interests, affinities, or location.

The web server may include a mail server or other messaging functionality for receiving and routing messages between the tech-bio exploration system 1204 and one or more client devices. An action logger may be used to receive communications from a web server about a user's actions on or off the tech-bio exploration system 1204. In conjunction with the action log, a third-party-content-object log may be maintained of user exposures to third-party-content objects. A notification controller may provide information regarding content objects to a client device. Information may be pushed to a client device as notifications, or information may be pulled from a client device responsive to a request received from the client device. Authorization servers may be used to enforce one or more privacy settings of the users of the tech-bio exploration system 1204. A privacy setting of a user determines how particular information associated with a user can be shared. The authorization server may allow users to opt in to or opt out of having their actions logged by the tech-bio exploration system 1204 or shared with other systems, such as, for example, by setting appropriate privacy settings. Third-party-content-object stores may be used to store content objects received from third parties. Location stores may be used for storing location information received from a client device associated with users.

In the foregoing specification, the invention has been described with reference to specific example embodiments thereof. Various embodiments and aspects of the invention(s) are described with reference to details discussed herein, and the accompanying drawings illustrate the various embodiments. The description above and drawings are illustrative of the invention and are not to be construed as limiting the invention. Numerous specific details are described to provide a thorough understanding of various embodiments of the present invention.

The present invention may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel to one another or in parallel to different instances of the same or similar steps/acts. The scope of the invention is, therefore, indicated by the appended claims rather than by the foregoing description. All changes that come within the meaning and range of equivalency of the claims are to be embraced within their scope.

What is claimed is:

1. A computer-implemented method comprising:
generating, utilizing a first classification model, a first set of perturbation classification scores from a first data modality;
generating, utilizing a second classification model, a second set of perturbation classification scores from a second data modality;
comparing the first set of perturbation classification scores with the second set of perturbation classification scores to determine a plurality of similarity measures; and
identifying, based on the plurality of similarity measures, pairs of data samples across the first data modality and the second data modality for a multi-modal machine learning model learning process utilizing the pairs of data samples.

2. The computer-implemented method of claim 1, wherein the first data modality comprises phenomic digital images and generating the first set of perturbation classification scores from the first data modality comprises generating, utilizing a phenomic image classification model, the first set of perturbation classification scores from a phenomic digital image of a cell exposed to a perturbation treatment.

3. The computer-implemented method of claim 1, wherein the second data modality comprises protein expression data and generating the second set of perturbation classification scores from the second data modality comprises generating, utilizing a protein expression classification model, the second set of perturbation classification scores from a protein expression measurement of a cell exposed to a perturbation treatment.

4. The computer-implemented method of claim 1, further comprising:
determining the plurality of similarity measures comprises determining a cross-modality distance within a feature space between a first perturbation of the first data modality and a second perturbation of the second data modality; and
identifying, utilizing a matching algorithm, the pairs of data samples across the first data modality and the second data modality based on the cross-modality distance.

5. The computer-implemented method of claim 1, further comprising:
generating, utilizing a multi-modal machine learning model from the multi-modal machine learning model learning process, multi-modal predictions for the pairs of data samples; and
modifying parameters of the multi-modal machine learning model utilizing a measure of loss determined based on the pairs of data samples.

6. The computer-implemented method of claim 5, further comprising utilizing the multi-modal machine learning model to:
generate protein expression data from phenomic digital images; or
generate phenomic digital images from protein expression data.

7. The computer-implemented method of claim 1, further comprising initiating the multi-modal machine learning model learning process utilizing the pairs of data samples by generating a matrix comprising entries that indicate probabilities of samples from the second data modality matching samples from the first data modality.

8. The computer-implemented method of claim 7, further comprising utilizing the matrix to initiate the multi-modal machine learning model learning process by:
determining, utilizing a loss function, a measure of loss by comparing a first sample from the first data modality and a second sample from the second data modality utilizing a probability from an entry of the matrix corresponding to the first sample from the first data modality and the second sample from the second data modality as a weight in the loss function; and
modifying parameters of a multi-modal machine learning model from the multi-modal machine learning model learning process based on the measure of loss determined utilizing the weight.

9. The computer-implemented method of claim 7, further comprising utilizing the matrix to initiate the multi-modal machine learning model learning process by determining a measure of loss by comparing a multi-modal prediction from the first data modality and two data samples for the second data modality according to corresponding entries in the matrix.

10. A system comprising:
at least one processor; and
at least one non-transitory computer-readable storage medium storing instructions that, when executed by the at least one processor, cause the system to:
generate, utilizing a first classification model, a first set of perturbation classification scores from a first data modality;
generate, utilizing a second classification model, a second set of perturbation classification scores from a second data modality;
compare the first set of perturbation classification scores with the second set of perturbation classification scores to determine a plurality of similarity measures; and
identify, based on the plurality of similarity measures, pairs of data samples across the first data modality and the second data modality for a multi-modal machine learning model learning process utilizing the pairs of data samples.

11. The system of claim 10, further comprising instructions that, when executed by the at least one processor, cause the system to generate the first set of perturbation classification scores from the first data modality by generating, utilizing a phenomic image classification model, the first set of perturbation classification scores from the first data modality comprising a phenomic digital image of a cell exposed to a perturbation treatment.

12. The system of claim 10, further comprising instructions that, when executed by the at least one processor, cause the system to generate the second set of perturbation classification scores from the second data modality by generating, utilizing a protein expression classification model, the second set of perturbation classification scores from the second data modality comprising a protein expression measurement of a cell exposed to a perturbation treatment.

13. The system of claim 10, further comprising instructions that, when executed by the at least one processor, cause the system to:
determine the plurality of similarity measures comprises determining a cross-modality distance within a feature space between a first perturbation of the first data modality and a second perturbation of the second data modality; and
identify, utilizing a matching algorithm, the pairs of data samples across the first data modality and the second data modality based on the cross-modality distance.

14. The system of claim 10, further comprising instructions that, when executed by the at least one processor, cause the system to:
generate, utilizing a multi-modal machine learning model from the multi-modal machine learning model learning process, multi-modal predictions for the pairs of data samples; and
modify parameters of the multi-modal machine learning model utilizing a measure of loss determined based on the pairs of data samples.

15. The system of claim 14, further comprising instructions that, when executed by the at least one processor, cause the system to utilize the multi-modal machine learning model by:
generating protein expression data from phenomic digital images; or
generating phenomic digital images from protein expression data.

16. The system of claim 10, further comprising instructions that, when executed by the at least one processor, cause the system to initiate the multi-modal machine learning model learning process utilizing the pairs of data samples by generating a matrix comprising entries that indicate probabilities of samples from the second data modality matching samples from the first data modality.

17. A non-transitory computer-readable medium storing instructions that, when executed by at least one processor, cause a computing device to:
generate, utilizing a first classification model, a first set of perturbation classification scores from a first data modality;
generate, utilizing a second classification model, a second set of perturbation classification scores from a second data modality;
compare the first set of perturbation classification scores with the second set of perturbation classification scores to determine a plurality of similarity measures; and
identify, based on the plurality of similarity measures, pairs of data samples across the first data modality and the second data modality for a multi-modal machine learning model learning process utilizing the pairs of data samples.

18. The non-transitory computer-readable medium of claim 17, further comprising instructions that, when executed by the at least one processor, cause the computing device to generate the first set of perturbation classification scores from the first data modality by generating, utilizing a phenomic image classification model, the first set of perturbation classification scores from the first data modality comprising a phenomic digital image of a cell exposed to a perturbation treatment.

19. The non-transitory computer-readable medium of claim 17, further comprising instructions that, when executed by the at least one processor, cause the computing device to generate the second set of perturbation classification scores from the second data modality by generating, utilizing a protein expression classification model, the second set of perturbation classification scores from the second data modality comprising a protein expression measurement of a cell exposed to a perturbation treatment.

20. The non-transitory computer-readable medium of claim 17, further comprising instructions that, when executed by the at least one processor, cause the computing device to:
generate, utilizing a multi-modal machine learning model from the multi-modal machine learning model learning process, multi-modal predictions for the pairs of data samples; and
modify parameters of the multi-modal machine learning model utilizing a measure of loss determined based on the pairs of data samples.
